(19) United States
(12) Patent Application Publication
(10) Pub. No.: US 2005/0287170 A1
(43) Pub. Date: Dec. 29, 2005

(54) Subunit vaccine against West Nile viral infection

(75) Inventors: Michael Lieberman, Honolulu, HI (US); Tom Humphreys, Honolulu, HI (US)

Correspondence Address: Paradise Patent Services, Inc.;Attn: George Darby, P.O. Box 893010, Mililani, HI, 96789-3010, US

(73) Assignee: HAWAII BIOTECH, INC.

(21) Appl. No.: 11/114,325

(22) Filed: Apr. 25, 2005

Related U.S. Application Data
(63) Continuation-in-part of application No. 10/730,776, filed on Dec. 8, 2003, now abandoned.
(60) Provisional application No. 60/493,312, filed on Aug. 6, 2003.
(60) Provisional application No. 60/432,865, filed on Dec. 11, 2002.

(51) Int. Cl. C12Q 1/70; C07H 21/04; A61K 39/12; C07K 14/005; C07K 16/08

(52) U.S. Cl. 424/204.1; 435/5; 530/350; 530/388.3; 435/69.3; 435/320.1; 435/325; 536/23.72

(57) ABSTRACT

An immunogenic composition is described that preferably contains a recombinantly produced form of truncated West Nile envelope glycoprotein and one or more adjuvants acceptable for use in the general population, including immunosuppressed, immunocompromised, and immunosenescent populations. The disclosed immunogenic compositions can further comprise a recombinantly produced non-structural (non-envelope) West Nile protein. An adjuvant typically comprises a saponin, saponin-based adjuvant (e.g., ISCOMATRIX® or GPI-0100), emulsion-based adjuvant (e.g., Co-Vaccine HT), or alum-based adjuvant. A pharmaceutically acceptable vehicle may also be included in the immunogenic composition.

### RELATED APPLICATIONS

This application is a continuation-in-part of U.S. non-provisional patent application Ser. No. 10/730,776, filed Dec. 8, 2003, which parent application is as of the application date hereof and claims the benefit of U.S. Provisional Patent Application No. 60/432,865, filed Dec. 11, 2002, and to U.S. Provisional Patent Application No. 60/493,312, filed Aug. 6, 2003, the disclosures and drawings of all of which prior applications are hereby incorporated by reference in their entirety.

### SEQUENCE LISTING

A Sequence Listing in ST.25 format on CD-ROM is appended to this application and fully incorporated herein by reference. The computer readable Sequence Listing appended to this application is identical to the written sequence listing contained herein. Notes on the Sequence Listing are appended hereto as Appendix A and fully incorporated herein.

### FIELD OF THE INVENTION

The invention relates to an immunogenic composition or vaccine designed to elicit an immunological response against flaviviral infection. Specifically, the immunogenic formulation comprises at least one recombinant flavivirus envelope (E) glycoprotein produced in a cellular production system and at least one adjuvant. One or more preferred adjuvants are selected from the group comprising saponins (e.g, GP-0100, ISCOMATRIX®), or derivatives thereof, emulsions alone or in combination with carbohydrates or saponins, aluminum-based formulations and oligodeoxyribonucleotides. The immunogenic formulation may also comprise at least one recombinant flavivirus non-structural protein, preferably NS1.

### DESCRIPTION OF RELATED ART

The family Flaviviridae includes the family prototype yellow fever virus (YF), the four serotypes of dengue virus (DEN-1, DEN-2, DEN-3, and DEN-4), Japanese encephalitis virus (JE), tick-borne encephalitis virus (TBE), West Nile virus (WN), Saint Louis encephalitis virus (SLE), and about 70 other disease causing viruses. Flaviviruses are small, enveloped viruses containing a single, positive-strand RNA genome. Ten gene products are encoded by a single open reading frame and are translated as a polyprotein organized in the order: capsid (C), “preMembrane” (prM, which is processed to “Membrane” (M) just prior to virion release from the cell), “envelope” (E), followed by non-structural (NS) proteins NS1, NS2a, NS2b, NS3, NS4a, NS4b and NS5 (reviewed in Chambers, T. J. et al., Annual Rev Microbiol (1990) 44:649-688; Henchal, E. A. and Putnak, J. R., Clin Microbiol Rev. (1990) 3:376-396). Individual flaviviral proteins are then produced through precise processing events mediated by host as well as virally encoded proteases.

The envelope of flaviviruses is derived from the host cell membrane, but contains the virally-encoded transmembrane envelope (E) glycoprotein. This E glycoprotein is the largest viral structural protein, and contains functional domains responsible for cell surface attachment and intraendosomal fusion activities. It is also a major target of the host immune system, inducing the production of virus neutralizing antibodies, which are associated with protective immunity.

Although the mode of flavivirus transmission and the pathogenesis of infection are quite varied among the different flaviviruses, dengue viruses serve as an illustrative example of the family. Dengue viruses are transmitted to man by mosquitoes of the genus Aedes, primarily A. aegypti and A. albopictus. The viruses cause an illness manifested by high fever, headache, aching muscles and joints, and rash (Gibbons, R. V. and D. W. Vaughn, British Medical Journal (2002) 324:1563-1566). Some cases, typically in children, result in a more severe form of infection, dengue hemorrhagic fever/dengue shock syndrome (DHF/DSS), marked by severe hemorrhage, vascular permeability, or both, leading to shock. Without diagnosis and prompt medical intervention, the sudden onset and rapid progression of DHF/DSS can be fatal.

Dengue viruses are the most significant group of arthropod-transmitted viruses in terms of global morbidity and mortality with an estimated one hundred million cases of dengue fever occurring annually including 250,000 to 500,000 cases of DHF/DSS (Gubler, D. J., Clin. Microbiol. Rev. (1998) 11:480-496; Gibbons, supra). With the global increase in population, urbanization of the population especially throughout the tropics, and the lack of sustained mosquito control measures, the mosquito vectors of dengue have expanded their distribution throughout the tropics, subtropics, and some temperate areas, bringing the risk of dengue infection to over half the world's population. Modern jet travel and human emigration have facilitated global distribution of dengue serotypes, such that multiple serotypes of dengue are now endemic in many regions. Accompanying this there has been an increase in the frequency of dengue epidemics and the incidence of DHF/DSS in the last 15 years. For example, in Southeast Asia, DHF/DSS is a leading cause of hospitalization and death among children (Gubler, supra; Gibbons and Vaughn, supra).

West Nile virus infection has become an emerging infectious disease in the United States. The virus infects birds, which serve as the natural reservoir for the virus, in addition to humans and horses, which are incidental hosts. It is an arthropod-borne virus transmitted by the Culex mosquito as well as at least 42 other species of mosquitoes. The first documented case of West Nile virus was found in the West Nile region of Uganda in 1937 (Smithburn et al., Am J Trop Med Hyg (1940) 20:471-492). It has since spread through the Middle East, Oceania, parts of Europe and Asia, and has recently emerged in the Americas. Since the first case of human infection in the U.S. was documented in New York City in 1999, the virus has rapidly spread throughout the east coast of the U.S. and has migrated westward. It has now been found in bird populations in 47 of the 48 states of the continental U.S., including the west coast (MMWR, vol. 52, 2003). Human cases of West Nile disease have been documented in 45 states, including California and the District of Columbia (CDC, 2004).

The majority of individuals infected with West Nile virus experience flu-like symptoms. However, a number of infected individuals will develop severe disease which carries a case-fatality rate of 3-15% and is highest among the elderly. In addition, in a high percentage of the non-fatal cases, permanent neurological disabilities result. In 2003, of 9,862 infected individuals, 2,866 (29%) had neuroinvasive disease (defined as West Nile meningitis, encephalitis and myelitis) and 264 died from the disease. Neuroinvasive complications have risen to 36% in 2004 (MMWR, vol. 53 Nov. 19, 2004). Recent studies have shown that recovery from viral infection requires significantly more time than originally thought. One study has concluded that the median recovery time was 60 days (Comment, Ann Inter. Med. (2004), 141:153) while another documented that only 37% of patients recovered completely after one year (Labowitz et al., Emerg. Inf. Dis. (2004) 10:1405-1411). The neurological damage done by the virus is slow to heal and, in some cases, is permanent. In recent years, some individuals have suffered from polio-like symptoms of acute flaccid paralysis. The clinical findings are significantly worse in elderly patients. In a study of a recent outbreak of West Nile infections in Israel, within the study group of 233 hospitalized patients, there was an overall case fatality rate of 14%. However, among patients aged 70 or older, the case fatality rate was 29% (Chowers et al., Emerg. Inf. Dis. (2001) 7:675-78). Similar findings were also reported from recent epidemics in Romania (Tsai et al., Lancet (1998) 352:767-771) and Russia (Platonov et al., Emerg. Inf. Dis. (1999) 7:128-32). Thus, there is significant morbidity and mortality associated with West Nile disease, especially among the elderly/immunosenescent, immunocompromised, and immunosuppressed populations.

Within the flaviviruses, all dengue viruses are antigenically related, but antigenic distinctions exist that define the four dengue serotypes. Infection of an individual with one serotype provides long-term immunity against reinfection with that serotype but fails to protect against infection with the other serotypes. In fact, immunity acquired by infection with one serotype may potentially enhance pathogenicity by other dengue serotypes.

This is particularly troubling as secondary infections with heterologous serotypes have become increasingly prevalent as the virus has spread, resulting in the co-circulation of multiple serotypes in many geographical areas and increased numbers of cases of DHF/DSS (Gubler, supra). Regardless of the mechanism for enhanced pathogenicity of a secondary, heterologous dengue infection, strategies employing a tetravalent vaccine should avoid such complications. Helpful reviews of the nature of the dengue disease, the history of attempts to develop suitable vaccines, structural features of flaviviruses in general, as well as the structural features of the envelope protein of flaviviruses are available (Halstead, S. B. Science (1988) 239:476-81; Brandt, E. E., J. Infect Disease (1990) 162:577-583; Chambers, supra; Mandl, C. W. et al., Virology (1989) 63:564-571; Henchal and Putnak, supra; Gubler, supra; Cardosa, M. J., Brit. Med. Bull. (1998) 54:395-405).

In contrast to dengue, West Nile virus has only been reported as a single serotype to date. At least two genotypes have been described (Bakonyi et al., Emer. Inf. Dis. (2005) 11:225) but thus far the differentiation between genotypes has not risen to the level of distinct serotypes. Thus a vaccine targeting the single defined serotype would be appropriate and likely sufficient. The West Nile envelope protein shares significant homology with the envelope proteins of other flaviviruses, particularly those of the other members of the Japanese encephalitis (JE) serocomplex: JE, St. Louis encephalitis (SLE), and Murray Valley (MV) viruses. Antibodies directed against particular epitopes contained within the envelope protein are capable of viral neutralization, i.e., the inhibition of virus infection of susceptible cells in vitro. In flaviviruses, serotype specific neutralizing epitopes have recently been mapped to E domain III (one of three domains of the envelope protein) using sets of monoclonal antibodies for dengue virus (Crill and Roehrig, J. Virol. (2001) 75:7769-7773), as well as JE (Lin and Wu, J. Virol. (2003) 77:2600-6) and West Nile viruses (Beasley and Barrett, J. Virol. (2002) 76:13097-13100). A high titer of viral neutralizing antibodies is generally accepted as the best in vitro correlate of in vivo protection against flaviviral infection and prevention of flavivirus induced disease (Markoff Vaccine (2000) 18:26-32; Ben-Nathan et al., J. Inf. Diseases (2003) 188:5-12; Kreil et al., J. Virol. (1998) 72:3076-3081; Beasley et al., Vaccine (2004) 22:3722-26). Therefore, a vaccine that induces high titer West Nile virus neutralizing responses will likely protect vaccinees against disease induced by West Nile virus.

Development of flavivirus vaccines has met with mixed success. A live attenuated vaccine for Yellow Fever virus has been available for many decades, while development of dengue and West Nile vaccines have significant challenges associated with them. While a significant amount of effort has been invested in developing candidate live-attenuated dengue vaccine strains, many strains tested have proven unsatisfactory (see, e.g., Eckels, K. H. et al., Am. J. Trop. Med. Hyg. (1984) 33:684-689; Bancroft, W. H. et al., Vaccine (1984) 149:1005-1010; McKee, K. T., et al., Am. J. Trop. Med. Hyg. (1987) 36:435-442). Despite this limited success, live attenuated candidate vaccine strains continue to be developed and tested (Bhamarapravati, N. et al., Bull. World Health Organ. (1987) 65:189-195; Hoke, C. H., Jr. et al., Am. J. Trop. Med. Hyg. (1990) 43:219-226; Angsubhakorn, S., et al., Southeast Asian J Trop. Med. Public Health (1994) 25:554-559; Dharakul, T. et al., J. Infect. Dis. (1994) 170:27-33; Edelman, R. et al., J. Infect. Dis. (1994) 170:1448-1455; Vaughn, D. W. et al., Vaccine (1996) 14:329-336; Bhamarapravati, N., and Sutee, Y., Vaccine (2000) Suppl 2:44-47; Kanesa-thasan, N. et al., Vaccine (2001) 19:3179-3188; Sabchareon, A. et al., Am. J. Trop. Med. Hyg. (2002) 66:264-272; Reviewed in Am. J. Trop. Med. Hyg. (2003) 69:1-60). Another approach to development of a live vaccine for dengue is a recombinant chimeric (intertypic) dengue vaccine (Bray, M. et al., J. Virol. (1996) 70:4162-4166; Chen, W., et al., J. Virol. (1995) 69:5186-5190; Bray, M. and Lai, C.-J., Proc. Natl. Acad. Sci. USA (1991) 88:10342-10346; Lai, C. J. et al., Clin. Diagn. Virol. (1998) 10:173-179). However, all of the live virus vaccine approaches remain plagued by difficulties in developing properly attenuated strains and in achieving balanced, tetravalent formulations.

Similarly, efforts to develop killed dengue vaccines have met with limited success. Primarily these studies have been limited by the inability to obtain adequate viral yields from cell culture systems. Virus yields from insect cells such as C6/36 cells are generally in the range of 10⁴ to 10⁵ pfu/ml, well below the levels necessary to generate a cost-effective killed vaccine. Yields from mammalian cells including LLC-MK2 and Vero cells are higher, but the peak yields, approximately 10⁸pfu/ml from a unique Vero cell line, are still lower than necessary to achieve a truly cost-effective vaccine product.

Similarly, there is currently no approved commercially available vaccine for prevention of West Nile virus infection in humans. There is also no specific therapy for disease, only symptomatic treatment. There are several candidate West Nile vaccines in various stages of research and development. These include: (i) a “naked” DNA vaccine encoding the prM and E genes (Chang et al., Ann. N.Y. Acad. Sci. (2001) 951:272-85); (ii) a live, attenuated dengue serotype 4-West Nile chimera (Pletnev et al., Proc. Natl. Acad. Sci. (2002) 99:3036-41); (iii) a live attenuated Yellow Fever-West Nile chimera (Monath et al., Curr. Drug Targets Infect. Disord. (2001) 1:37-50); (iv) a recombinant envelope protein vaccine expressed in E. coli (Wang et al., J. Immunol. (2001) 167:5273-77); (v) a live, attenuated West Nile (veterinary) vaccine (Lustig et al., Viral Immunol. (2000) 13:401-410); (vi) baculovirus produced prM and E containing virus like particles (Qiao et al., J. Inf. Dis. (2004) 190:2104-8); and (vii) a formalin-inactivated West Nile (veterinary) vaccine (Tesh et al., Emerg. Inf. Dis. (2002) 8:1392-7). Associated with each of these candidate vaccines are intrinsic difficulties. Safety concerns, of course, are paramount with all live viral vaccines, particularly in the case of a virus disease with relatively low prevalence, in which the vaccine is given to healthy subjects. Under-attenuation of the virus may result in disease manifestation, whereas over-attenuation may abrogate vaccine efficacy. Also, reversion to wild type or mutation to increased virulence (or decreased efficacy) may occur. Moreover, even if properly attenuated, live viral vaccines are contra-indicated for specific patient populations such as infant, elderly/immunosenescent, immunocompromised, and immunosuppressed populations, as well as particular segments of the normal population, such as pregnant women. For instance, there is mounting concern related to unanticipated deaths linked to administration of live attenuated Yellow Fever vaccine to healthy elderly subjects (CDC—MMWR 51:1-10, Yellow Fever Vaccine Recommendations of the Advisory Committee on Immunization Practices (ACIP) 2002). Inactivated whole virus vaccines may present production problems at commercial scale in terms of growth of the virus to sufficiently high titers for economical yield, as well as hazardous containment issues for large scale growth of non-attenuated live virus. Naked DNA vaccines are unproven for any infectious disease at this time, and the issue of potential immunopathology due to the induction of an autoimmune reaction to the DNA over the long term is unresolved. Finally, the expression of recombinant flaviviral proteins in bacterial or baculovirus systems has often resulted in aberrant tertiary and/or quaternary structure of the expressed proteins resulting in poor yields and low immunogenicity.

In the absence of effective live attenuated or killed dengue or West Nile vaccines, a significant effort has been invested in the development of recombinant subunit vaccines. Many of the vaccine efforts that use a recombinant DNA approach have focused on the E glycoprotein. This glycoprotein is a logical choice for a subunit vaccine as it plays a central role in the biology and the host immune response to the virus. The E glycoprotein is exposed on the surface of the virus, binds to the cell receptor, and mediates fusion (Chambers, supra). It has also been shown to be the primary target for the neutralizing antibody response (Mason, P. W., J Gen Virol (1989) 70:2037-2048). Monoclonal antibodies directed against purified flaviviral E proteins are neutralizing in vitro and some have been shown to confer passive protection in vivo (Henchal, E. A. et al., Am. J. Trop. Med. Hyg. (1985) 34:162-169; Heinz, F. X. et al., Virology (1983) 130:485-501; Kimura-Kiroda, J. and Yasui, K., J. Immunol. (1988) 141:3606-3610; Trirawatanapong, T. et al., Gene (1992) 116:139-150).

While many heterologous expression systems have been developed and shown to be effective for production of certain recombinant products, not all expression systems are effective for producing all recombinant products. In fact, despite the fact that a system may be reported to be effective for production of one recombinant protein, predictions on efficacy of expression of other recombinant products do not always hold. In particular, efficient expression of conformationally relevant recombinant flavivirus E has remained elusive. A wide variety of expression systems ranging from bacterial, fungal, and insect to mammalian systems have failed to efficiently produce conformationally relevant flavivirus E in significant quantities, highlighting the highly empirical nature of efficient heterologous gene expression.

Much progress in the analysis and understanding of the immune response to foreign antigens has been made in the last decade or two, particularly in the realm of cellular immunology. The delineation of subsets of lymphocytes with distinct functional properties and the characterization of the interactions between these subsets of cells has provided detailed mechanistic explanations for the overall functioning of the immune system. One central paradigm that has emerged revolves around the description of two classes of T “helper” lymphocytes, termed “Th1” and “Th2” cells (Table 1). These two classes of T cells are primarily distinguished by the pattern of cytokine expression elaborated by each. The cytokines produced by Th1 cells (IFN-γ, IL-2, TNF-β) tend to promote the cellular immune effector response required to combat parasitic, fungal, and intracellular viral agents as well as production of antibody subclasses associated with important effector mechanisms such as virus neutralization capacity (Moingeon, P., J. Biotechnol. (2002) 98:189-198; Azzari C. et al., (1987) Pediatr. Med. Chir. 9:391-6; Smucny, J. et al., (1995) Am. J. Trop. Med. Hyg. 53:432-7). The cytokines produced by Th2 cells (IL-4, IL-5, IL-6, IL-10, IL-13), tend to promote antibody synthesis effective in controlling extracellular bacterial pathogens. The balance between Th1 and Th2 cytokines is a dynamic one, because of the fact that Th1 cytokines tend to inhibit the production of Th2 cytokines in vivo, and vice versa. Thus, a viral vaccine capable of stimulating a “Th1” type immune response would reasonably be expected to be more efficacious in protection against infection than a vaccine eliciting only an antibody response.

TABLE 1
T helper type 1 (Th1) and T helper type 2 (Th2) lymphocytes

| | TH1 | |
|---|---|---|
| CHARACTERISTIC | LYMPHOCYTES | TH2 LYMPHOCYTES |
| Cytokines produced | IFN-γ, TNF-β, IL-2 | IL-4, IL-5, IL-6, IL-10, |
| | | IL-13 |
| | | (IL-4 is particularly |
| | | important for IgE |
| | | synthesis) |
| Type of associated | Cell-Mediated | Humoral |
| immune response | | |
| Associated antibody | IgG_{2A} (mouse) | IgG₁ (mouse) |
| isotypes | IgG₁, IgG₃ (human) | IgG₂, IgG₄ (human), IgE |

Suppression or impairment of either arm of the immune system can lead to increased susceptibility or severity of disease induced by infectious agents (e.g. opportunistic infections). In “immunosuppressed” individuals, the immune response is prevented or diminished (e.g., by administration of radiation, antimetabolites, antilymphocyte serum, or specific antibody). “Immunocompromised” or “immunodeficient” individuals have their immune system attenuated (e.g., by malnutrition, irradiation, cytotoxic chemotherapy, or diseases such as cancer or AIDS), Recent advances in understanding of aging and immunology have suggested that elderly subjects also show a decreased immunoresponsiveness, sometimes referred to as immunosenescence (Pawelec, Biogerontology (2003) 4:167-70; Mishto et al., Ageing Res. Rev. (2003) 2:419-32; McElhaney, Conn. Med. (2003) 67:469-74; Pawelec et al., Front. Biosci. (2002) 7:d1056-183; Katz et al., Immunol. Res. (2004) 29:113-24). Elderly and infant subjects (especially, non-suckling infants) are also recognized to be more susceptible to infectious diseases (e.g., influenza infection—Katz et al., supra) consistent with an impaired or immature immune system. Immunosuppressed, immunocompromised, immunosenescent, and non-suckling infant populations (collectively, the “immunodeficient population”) are at particular risk for many infectious diseases, but concomitantly are too vulnerable to the effects of reversion or mutation of attenuated live virus vaccines, and therefore are an important target audience for vaccine development. However, the fact that the individuals have some sort of immune impairment makes the challenge for developing an immunogenic and protective vaccine for the immunodeficient population particularly difficult.

In addition to the challenges linked to the ability to induce an appropriate and potent immune response in the immunodeficient population, an additional hurdle lies in the safety of classical vaccine approaches for this target population. By definition, live attenuated vaccines replicate in the vaccinee. However the safety profile of the live attenuated virus is generally established in healthy adults or children with intact immune systems which regulate the replication of the vaccine entity. In the immunodeficient population, this regulation may be absent and the attenuated vaccine may replicate out of control and induce significant disease. Recent reports of death following administration of live attenuated Yellow Fever vaccine to healthy elderly subjects highlight the risks associated to vaccination of this population (Yellow Fever Vaccine Recommendations of the Advisory Committee on Immunization Practice ACIP 2002—MMWR Nov. 8, 2002; Lawrence et al., Commun. Dis. Intell. (2003) 27:307-323; Leder et al., Clin. Infect. Dis. (2001) 33:1553-66).

In the case of West Nile virus induced disease, the epidemiology shows that the disease is most prevalent and most severe in elderly subjects (Chowers et al., supra; Tsai et al., supra; Platonov et al., supra). Therefore development of a West Nile vaccine which could be effective in elderly and other members of the immunodeficient population represents a significant challenge. Live attenuated vaccine approaches are unlikely to have an appropriate safety profile in a key target population, the immunodeficient. Thus alternative approaches that can overcome the immune limitations of the immunodeficient population and remain safe in this population are highly desirable.

Adjuvants are materials that increase the immune response to a given antigen. Since the first report of such an enhanced immunogenic effect by materials added to an antigen (Ramon, G., Bull. Soc. Centr. Med. Vet. (1925) 101:227-234), a large number of adjuvants have been developed, but only calcium and aluminum salts are currently licensed in the United States for use in human vaccine products. Numerous studies have demonstrated that other adjuvants are significantly more efficacious for inducing both humoral and cellular immune responses. However, most of these have significant toxicities or side-effects which make them unacceptable for human and veterinary vaccines. In fact, even aluminum hydroxide has recently been associated with the development of injection site granulomas in animals, raising safety concerns about its use. Because of these problems, significant efforts have been invested in developing highly potent, but relatively non-toxic adjuvants. A number of such adjuvant formulations have been developed and show significant promise (Cox, J. C. and Coulter, A. R., Vaccine (1997) 15:248-256; Gupta, R. K. and Siber, G. R., Vaccine (1995) 13:1263-1276), especially in combination with recombinant products. Several of these modern adjuvants are being tested in preclinical and clinical trials designed to examine both efficacy and safety.

The main modes of action of adjuvants include (i) a depot effect, (ii) direct immunomodulation through interaction with receptors, etc., on the surface of immune cells and (iii) targeting antigens for delivery into specific antigen-presenting cell populations (e.g., through the formation of liposomes or virosomes). The depot effect results from either the adsorption of protein antigens onto aluminum gels or the emulsification of aqueous antigens in emulsions. In either case this results in the subsequent slow release of these antigens into the circulation from local sites of deposition. This prevents the rapid loss of most of the antigen that would occur by passage of the circulating antigen through the liver. Immunomodulation involves stimulation of the “innate” immune system through interaction of particular adjuvants with cells such as monocytes/macrophages or natural killer (NK) cells. These cells become activated and elaborate proinflammatory cytokines such as TNF-α and IFN-γ, which in turn stimulate T lymphocytes and activate the “adaptive” immune system. Bacterial cell products, such as lipopolysaccharides, cell wall derived material, DNA, or oligonucleotides often function in this manner (Krieg, A. M. et al., Nature (1995) 374:546; Ballas, Z, J, et al., J. of Immunology (2001) 167:4878-4886; Chu, R. S., et al., J. Exp. Med. (1997) 186:1623; Hartmann, G. and Krieg, A., J. Immunol. (2000) 164:944-952; Hartmann, G., et al., J. of Immunol. (2000) 164:1617-1624; Weeratna, R. D. et al., Vaccine (2000) 18:1755-1762; U.S. Pat. Nos.: 5,663,153; 5,723,335; 6,194,388; 6,207,646; 6,214,806; 6,218,371; 6,239,116; 6,339,068; 6,406,705; 6,429,199). Targeting of antigens to (and within) antigen presenting cells is accomplished through the delivery and fusion of antigen bearing vehicles (e.g., liposomes or virosomes) with antigen presenting cells, thereby delivering the antigen into the intracellular pathways necessary for presentation of antigen in the context of MHC Class I and/or II molecules (Leserman, L., J. Liposome Res. (2004) 14:175-89; Bungener et al., Vaccine (2005) 23:1232-41).

Through these different modes of action it may be possible to overcome the immune limitations found in the immunodeficient population. However, the specific immune deficiency and appropriate mechanism for overcoming the specific deficiency in the elderly and other members of the immunodeficient population is not well understood and therefore the appropriate solution is not predictable from the existing art. For example, efforts to overcome these difficulties and develop improved influenza vaccines for the elderly have taken various approaches (Guebre-Xabier et al., J. Virol. (2004) 78:7610-8; Frech et al., Vaccine (2005) 23:946-50; Ben-Yehuda et al., Vaccine (2003) 21:3169-78; Podda and Del Giudice, Expert Rev. Vaccines (2003) 2:197-203; Banzhoff et al., Gerontology (2003) 49:177-84; Gluck and Metcalf, Vaccine (2002) 20:B10-6; Ennis et al., Virology (1999) 259:256-61; Windon et al., Vaccine (2001) 20:490-7) with mixed results. However, even for those products which claim to have augmented the immune response, the clinical impact of the effect has been questioned: in side by side comparisons of adjuvanted vaccine products versus classical influenza vaccines, the adjuvanted products failed to show an added benefit (Ruf et al., Infection (2004) 32:191-8; Prescrire Int. (2004) 13:206-8). This highlights the difficulties faced in developing safe, effective flaviviral vaccines, especially vaccines for the immunodeficient population and other populations in which adjuvants make a substantial difference in immunogenicity and protection.

The technical problem to be solved by the invention is the discovery of flavivirus antigen/adjuvant combinations that simultaneously satisfy three conditions; an antigen/adjuvant combination must (1) induce relevant protective immune responses in vaccinated individuals, (2) overcome the immune limitations of the immunodeficient population (especially the elderly), and (3) maintain an acceptable safety profile. This represents a significant challenge in flavivirus vaccine development, particularly West Nile Virus vaccine development, and to date no vaccine approach has been shown to adequately address all aspects of this technical problem. There is very high, unmet and growing demand for a solution. The demand grows each summer, as the prevalence of West Nile viral infection spreads.

### SUMMARY OF THE INVENTION

The inventors have identified unique combinations of antigen and adjuvant that induce relevant protective immune responses in vaccinated individuals and that have shown an acceptable safety profile in several host species. These unique formulations depend upon a novel, properly folded recombinant subunit protein (“West Nile 80E”) combined with one or more adjuvants, such as saponins, emulsions, and alum-based formulations. These antigen/adjuvant combinations (1) induce relevant, protective immune responses, such as virus neutralizing antibody and cell mediated responses, (2) overcome the immune limitations of the immunodeficient population (especially the elderly), and (3) maintain an acceptable safety profile. The disclosed invention provides immunogenic compositions containing as active ingredients recombinantly-produced forms of truncated flavivirus envelope glycoproteins, and optionally, non-structural (“NS”) proteins. A preferred embodiment of the disclosed invention comprises the recombinant truncated envelope protein of West Nile virus as active ingredient. A preferred embodiment of the disclosed invention alternatively includes a dimeric form of the recombinant truncated flavivirus envelope protein. A preferred embodiment of the disclosed invention also includes an adjuvant, such as a saponin or a saponin-like material (e.g., GPI-0100, ISCOMATRIX®), alum-based formulations (e.g., Alhydrogel), or emulsion-based formulations (e.g., Co-Vaccine HT), either alone or in combination with other immunostimulants and adjuvants, as a component of the immunogenic formulations described herein. Typically, the disclosed immunogenic formulations are capable of eliciting the production of neutralizing antibodies against flaviviruses, in particular West Nile virus, and stimulating cell-mediated immune responses.

Other aspects of this invention include use of a therapeutically effective amount of the immunogenic composition in an acceptable carrier for use as an immunoprophylactic against flavivirus infection and a therapeutically effective amount of the immunogenic composition in an acceptable carrier as a pharmaceutical composition.

Other aspects of this invention include use of the recombinant truncated flavivirus envelope protein as a diagnostic reagent or in the preparation of a diagnostic kit.

Other aspects of the disclosed invention include use of the recombinant truncated flavivirus envelope protein to produce transformed immune B cells, antibodies, and hybridomas for generation of antibody or antibody-derived reagents for use as prophylactic or therapeutic treatments for flavivirus infection. Another aspect of the disclosed invention include use of the recombinant truncated flavivirus envelope protein for identification or development of small molecule antivirals. The disclosed immunogenic formulations induce higher titer virus neutralizing antibodies, and induce more potent cell-mediated immune responses, in comparison to conventional formulations.

### BRIEF DESCRIPTION OF THE DRAWINGS

FIG. 1A. Coomassie blue stained SDS-PAGE of West Nile 80E protein expressed by Drosophila S2 cells under non-reducing conditions. Lane1) Spinner Culture #1 of cell line WN-80E-1 harvested Feb. 19, 2003, Lane 2) Spinner Culture #2 of cell line WN-80E-1 harvested Feb. 10, 2003, Lane 3) Culture of a dengue transformant cell line. The migration of the West Nile 80E is faster than the dengue 80E due to differences in glycosylation and tertiary structure (samples are non-reduced).

FIG. 1B. Western blot of duplicate SDS-PAGE gel seen in FIG. 1A. The blot was probed with a commercially available West Nile rabbit polyclonal antibody from BioReliance (Rockville, Md.). This antibody cross-reacts slightly with the Dengue 80E.

FIG. 2A. Coomassie blue stained SDS-PAGE of West Nile NS1 protein expressed by Drosophila S2 cells under reducing (Lanes 1 and 2) and non-reducing conditions (Lanes 3 and 4). Lanes 1 and 3) Spinner Culture #1 of cell line WN-NS1-5 harvested Jul. 6, 2003, Lanes 2 and 4) Spinner Culture #2 of cell line WN-NS1-5 harvested Jul. 6, 2003.

FIG. 2B. Western blot of duplicate SDS-PAGE gel seen in FIG. 2A. The blot was probed with the mouse monoclonal 7E11. The two approximately 40 kD bands of WN-NS1 are two different glycoforms of the NS1 protein. The higher MW reactive band at about 80 kD in lanes 3 and 4 is a dimer. The 7E11 antibody reacts more strongly with reduced than non-reduced NS1.

FIG. 3. Coomassie stained SDS-PAGE gel (A) and Western blot (B) of purified West Nile 80E. Both samples were run under non-reducing conditions on 10% gels. The Western blot was developed using a rabbit polyclonal antisera developed against formalin inactivated dengue virus. The sizes of the molecular weight markers (in kD) are indicated to the left of the gel and blot. The sample loadings (in μg) are presented at the top of each.

FIG. 4 Coomassie stained SDS-PAGE gel (A) and Western blot (B) of purified West Nile NS1. Both samples were run under non-reducing conditions on 10% gels. The Western blot was developed using a rabbit polyclonal antisera developed against purified dengue NS1. The sizes of the molecular weight markers (in kD) are indicated to the left of the gel and blot. The sample loadings (in μg) are presented at the top of each.

FIG. 5. Lymphoproliferative Responses Induced in Mice by Adjuvanted Formulations. Bar graph demonstrating the effect of WN 80E dose (in presence of WN NS1) and adjuvant on the in vitro lymphoproliferative response by splenocytes from mice immunized with the vaccine of the invention and stimulated in vitro with the vaccine antigens. Lymphoproliferative response is expressed as stimulation index—counts per minute in stimulated cells divided by counts per minute in unstimulated controls.

FIG. 6. Interferon Gamma Responses Induced in Mice by Adjuvanted Formulations. Bar graph demonstrating the effect of WN 80E dose (in presence of WN NS1) and adjuvant on the production of interferon-γ (IFN-γ) in vitro by splenocytes from mice immunized with the vaccine of the invention and stimulated in vitro with the vaccine antigens. IFN-γ expressed in nanograms/ml.

FIG. 7. Interleukin 5 Responses Induced in Mice by Adjuvanted Formulations. Bar graph demonstrating the effect of WN 80E dose (in presence of WN NS1) and adjuvant on the production of interleukin-5 (IL-5) in vitro by splenocytes from mice immunized with the vaccine of the invention and stimulated in vitro with the vaccine antigens. IL-5 expressed in nanograms/ml.

### DESCRIPTION OF THE PREFERRED EMBODIMENTS

The invention described herein provides a subunit flavivirus immunogenic formulation that is produced and secreted using a recombinant expression system and combined with one or more adjuvants in immunogenic formulations. The disclosed immunogenic formulations are effective in inducing a strong virus neutralizing antibody response to Flaviviruses as well as stimulating cell-mediated immune responses to the viruses.

The inventors and their colleagues have successfully developed a proprietary method of expression in Drosophila cell systems that produces recombinant envelope proteins from flaviviruses such as dengue serotypes 1-4, West Nile, Japanese Encephalitis, hepatitis C, and Tick Borne Encephalitis virus (Cuzzubbo et al., Clin. Diagn. Lab. Immunol. (2001) 8:1150-55; Modis et al., Proc. Natl. Acad. Sci. (2003) 100:6986-91; Modis et al., Nature (2004) 427:313-9). These proteins are typically truncated at the C-terminus, leaving 80% of the native envelope protein (“80E”). The scope of the truncated proteins used in the invention includes any E protein secretable by the expression system, i.e., up to approximately 90% of the native envelope protein. The preferred truncation (that which produces 80E) deletes the membrane anchor portion (approximately the first 10% of E, starting from the carboxy end) of the protein, thus allowing it to be secreted into the extracellular medium, facilitating recovery. “Expression” and “to express” are synonymous with “secretion” and “to secret” as used herein. Cloning and expressing 80% or more but less then 90% of the E protein includes all (if 90% E) or part (if between 80% and 90% E) of the “stem” portion of the E protein that links the 80E portion with the membrane anchor portion; the stem portion does not contain notable antigenic epitopes and therefore is not included in the preferred antigen, 80E. More than 90%, but less than 100%, of the E protein can be cloned and secreted, i.e., the protein can be 90%+ in length, carboxy truncated, and can include a portion of the membrane spanning domain so long as the truncated E protein is secretable. However, the stem and partial membrane spanning domain portions do not contain notable antigenic epitopes and inclusion of any of the membrane spanning domain reduces yields; therefore the stem and partial membrane spanning domain portions are not included in the preferred antigen, 80E. “Secretable” means able to be secreted, and typically secreted, from the transformed cells in the expression system. Furthermore, the expressed proteins have been shown to be properly glycosylated and to maintain native conformation as determined by reactivity with conformationally sensitive monoclonal antibodies, 4G2 and 9D12, (Coller, BG, Clements, DE, Bignami, GS, et. al., Hawaii Biotech, unpublished data), and x-ray crystallography structure determination (Modis et al., supra). The proteins are potent immunogens when administered in combination with modern adjuvants and have been shown to induce protective efficacy in a small animal model for West Nile (see Examples below) and a non-human primate model for dengue (Putnak et al., submitted to Vaccine). Thus the inventors have found a novel solution to a key technical problem: the efficient production of conformationally relevant West Nile envelope protein which serves as a potent immunogen in vaccinated subjects, even those in the immunodeficient population.

In accordance with the invention, the disclosed immunogenic compositions may include an adjuvant. A preferred adjuvant is a saponin or a saponin-derivative or saponin-like substance (e.g., GPI-0100, ISCOMATRIX®) (saponin-derivative and saponin-like substances are collectively referred to herein as “saponin-based”), alum-based adjuvants (e.g., Alhydrogel), or emulsion-based adjuvants (e.g., Co-Vaccine HT).

The antigens used in the disclosed immunogenic compositions typically comprise a truncated flavivirus envelope protein alone or in combination with a non-structural protein. For example, a preferred immunogenic composition comprises a Drosophila cell-expressed envelope protein (preferably 80E). The envelope protein subunit (i.e., truncated, secretable E protein) from the WN virus is used in the WN vaccine composition. Envelope proteins subunits from other flaviviruses, such as Japanese encephalitis virus (JE), tick-borne encephalitis virus (TBE), dengue (DEN), and Saint Louis encephalitis virus (SLE), can be used as replacement or additional antigens in the disclosed invention.

An optional recombinant flavivirus non-structural protein can be included in the disclosed immunogenic composition. For example, a Drosophila cell-expressed non-structural protein (preferably NS1), preferably from the homologous flavivirus is included in the disclosed immunogenic compositions. Inclusion of these components typically results in an exceptionally potent vaccine formulation.

The combination of viral subunit E, with or without non-structural proteins, and with one or more adjuvants, induces very high titer neutralizing antibodies in mice. For example, certain combinations of a saponin-like material, preferably GPI-0100 or ISCOMATRIX®, as adjuvant with a given recombinant antigen yields a higher titer of virus neutralizing antibodies than the antigen alone. The cell-mediated response (correlated with the production of IFN-γ from immune splenocytes by antigenic stimulation in vitro) is significantly enhanced when these adjuvants are used with the recombinant protein(s). Examples illustrating the efficacy of the unique combination are contained herein below.

Envelope Protein Subunits (80E)

In the most preferred embodiment of the invention, the recombinant protein components of the flavivirus vaccine formulations described herein are produced by a eukaryotic expression system, Drosophila melanogaster Schneider 2 (S2) cells (Johansen, H. et al., Genes Dev. (1989) 3:882-889; Ivey-Hoyle, M., Curr. Opin. Biotechnol. (1991) 2:704-707; Culp, J. S., et al., Biotechnology (NY) (1991) 9:173-177). This method of expression successfully produces recombinant envelope proteins from Flaviviruses, such as dengue serotypes 1-4, WN, and Japanese encephalitis virus (JE). These proteins are truncated at the C-terminus, leaving approximately 80% of the native envelope protein (80E). The truncation deletes the membrane anchor of the protein, thus allowing it to be secreted into the extracellular medium, facilitating recovery; the truncation also deletes the stem portion, which has little immunogenic effect. Furthermore, the expressed proteins have been shown to be properly glycosylated and to maintain native conformation as determined by reactivity with conformationally sensitive monoclonal antibodies (e.g. 4G2, see example 2) and X-ray crystallographic analysis (Modis et al., supra; Modis et al., supra). The amino acid sequence listing of WN 80E is SEQ ID:1. The nucleotide sequence listing, including leading and trailing nucleotides (collectively, “bookends”) used in cloning, that encodes WN 80E is SEQ ID:2. The nucleotide sequence listing, without “bookends” used in cloning, that encodes WN 80E is SEQ ID:3. The amino acid sequence listing of WN NS1 is SEQ ID:4. The nucleotide sequence listing, including “bookends” used in cloning, that encodes WN NS1 is SEQ ID:5. The nucleotide sequence listing, without “bookends” used in cloning, that encodes WN NS1 is SEQ ID:6. The sequence listing portion of the information recorded in computer readable form and submitted with this application is identical to the written sequence listing below.

In another embodiment of the invention, 80E is defined more broadly as an envelope protein subunit that comprises six disulfide bridges at Cys1-Cys2, Cys3-Cys8, Cys4-Cys6, Cys5-Cys7, Cys9-Cys10 and Cys11-Cys12; wherein the polypeptide has been secreted as a recombinant protein from Drosophila cells; and wherein the polypeptide generates neutralizing antibody responses to a homologous strain of a species of Flavivirus.

In a more preferred embodiment, the envelope protein subunit further comprises a hydrophilicity profile characteristic of a homologous 80% portion of an envelope protein (80E) starting from the first amino acid at the N-terminus of the envelope protein of a strain of a species of Flavivirus. In other words, amino acids can be substituted in the sequence comprising 80E so long as the hydrophilicity profile and immunogenicity are unchanged.

The immunogenicity and protective efficacy of such truncated E proteins have also been amply demonstrated in animal models (U.S. Pat. Nos. 6,136,561; 6,165,477; 6,416,763; 6,432,411; Jan, L., et al., Am. J. Trop. Med. Hyg., 48(3), (1993) pp. 412-423; Men, R. et al., J. Virol (1991) 65:1400-1407).

As previously described (Ivy et al., U.S. Pat. No. 6,136,561; Ivy et al., U.S. Pat. No. 6,165,477; McDonnell et al., U.S. Pat. No. 6,416,763; Ivy et al., U.S. Pat. No. 6,432,411, which patents are all fully incorporated herein by reference) and, used herein, “80E” in one instance refers to a polypeptide that spans a flavivirus envelope protein, preferably one starting from the N-terminal amino acid of the envelope protein and ending at an amino acid in the range of the 395^{th} to 401^{st} amino acid, for example, such 80E can be the polypeptide comprising amino acids 1 to 401 of WN virus or 1 to 395 of DEN type 2.

Preferably, the WN envelope protein subunit is a portion of the WN envelope protein that comprises approximately 80% of its length starting from amino acid residue 1 at its N-terminus and which portion has been recombinantly produced and secreted from Drosophila cells. In another embodiment, 80E is at least 80%, or 85%, or 90% or 95% homologous over the entire sequence relative to native flavivirus 80E. More preferably, 80E is derived from homologs or variants as described above, e.g., all West Nile variants as well as any serotypes of: Japanese encephalitis virus (JE), Tick-borne encephalitis virus (TBE), dengue virus (DEN), Saint Louis encephalitis virus (SLE), and the family prototype, Yellow fever virus (YF). The 80E proteins preferably are produced from vectors containing the DNA encoding the WN virus prM as a fusion with 80E. The fusion protein is processed by cellular enzymes to release the mature 80E proteins.

In one embodiment, the immunogenic composition comprises the envelope protein subunit derived from WN virus. Preferably, the 80E subunit from WN virus is purified by immunoaffinity chromatography (IAC) using a monoclonal antibody (4G2) as previously described (Ivy et al., U.S. Pat. No. 6,432,411, example 9).

Dimeric 80E

Numerous studies have demonstrated that immunogenicity is directly related both to the size of the immunogen and to the antigenic complexity of the immunogen. Thus, in general, larger antigens make better immunogens. The native form of E protein found on the surface of the flavivirus virion is a homodimer (Rey F. A. et al., Nature (1995) 375:291-298). The recombinant WN 80E protein discussed above is monomeric and therefore is not identical to the natural viral E protein. Thus, in an attempt to produce a protective recombinant flavivirus immunogenic formulation, preferably an immunogenic formulation protective against DEN virus infection, with enhanced immunogenicity, dimerized versions of the dengue 80E proteins were produced by genetic engineering techniques (Peters et al., U.S. Pat. No. 6,749,857). In a preferred embodiment, the envelope protein subunit from WN is a dimer.

The modifications that can be made to the 80E products by addition of carboxy-terminal sequences encoding flexible linkers, and leucine zipper domains or four helix bundle domains, designed to enhance the dimerization of the 80E molecules, are described in detail below. All of these dimeric 80E proteins are produced from vectors containing the DNA encoding the flavivirus prM as a fusion with mature proteins resulting in secretion of the processed, mature dimeric 80E proteins from which the prM protein has been removed.

Three basic approaches have been disclosed in U.S. Pat. No. 6,749,857 to construct dimeric 80E molecules. The first approach involves using tandem copies of 80E covalently attached to each other by a flexible linker. In a preferred embodiment, “Linked 80E Dimer” refers to a polypeptide which encodes WN 80E—GGGSGGGGSGGGTGGGSGGGSGGGG—WN 80E. The stretch of amino acids covalently linking the two copies of WN 80E is designed to serve as a flexible tether allowing the two 80E molecules to associate in native head-to-tail dimeric orientation while maintaining their covalent attachment to each other. “Linked 80E Dimer” also refers to the corresponding peptide region of the envelope protein of others WN homologs and to any naturally occurring variants, as well as corresponding peptide regions of the E protein of other Flaviviruses. For example, serotypes of JE, TBE, DEN, SLE and YF are included.

It would be readily apparent to one of ordinary skill in the art to select other linker sequences as well. The portion of present invention directed to dimeric molecules is not limited to the specific disclosed linkers, but, to any amino acid sequence that would enable the two 80E molecules to associate in native head to tail dimeric orientation. The linkage of the soluble monomers results in a local concentration of monomers that is sufficiently high to favor the association of the conformationally correct monomers in the native quaternary head-to-tail dimeric conformation.

The second approach involves addition of a carboxy-terminal leucine zipper domain to monomeric 80E to enhance dimerization between two 80E-leucine zipper molecules. Two versions of this approach have been adopted. One version includes a disulfide bond linking the leucine zipper domains resulting in a covalently linked dimer product, while the other is based on the non-covalent association of the leucine zipper domains. As used herein “80E ZipperI” refers to a polypeptide that, in association with another polypeptide, produces a non-covalently linked dimer, and preferably refers to a polypeptide which encodes WN 80E—GGGSGGGGSGGGTGGGSGGGSPRMKQLEDKVEELLSKNYHLENEVARLKKLVGER. The first 22 amino acids extending after the carboxy terminus of 80E serve as flexible tether between 80E and the adjacent leucine zipper domain. The leucine zipper domain is designed to dimerize with the identical sequence from another 80E Zipper molecule. The formation of a non-covalently linked leucine zipper will enhance the dimerization of the 80E molecules, which may associate in native head to tail conformation by virtue of the flexible linker connecting the 80E molecules with the leucine zipper domain. “80E ZipperI” also refers to the corresponding peptide region of the envelope protein of other WN homologs or any naturally occurring variants, as well as corresponding peptide regions of the E protein of other flaviviruses, for example, any serotypes of JE, TBE, DEN, SLE and YF. The association between leucine zipper domains results in a local concentration of 80E monomers that is sufficiently high to favor the association of the conformationally correct monomers in the native quaternary head-to-tail dimeric conformation.

It would be readily apparent to one of ordinary skill in the art to select other leucine zipper sequences as well. The present invention is not limited to the specific disclosed leucine zipper sequences, but to any amino acid sequences that would enable the dimerization between identical sequences from another 80E molecule with a flexible tether.

As used herein “80E ZipperII” refers in one instance to a polypeptide that, in association with another polypeptide, produces a covalently linked dimer and preferably to a polypeptide which encodes WN 80E—GGGSGGGGSGGGTGGGSGGGSPRMKQLEDKVEELLSKNY HLENEVARLKKLVGERGGCGG. The first 22 amino acids extending after the carboxy terminus of 80E serve as flexible tether between 80E and the adjacent leucine zipper domain. In one preferred embodiment, the method of making a “ZipperII” dimer involves addition of a carboxy-terminal peptide linker (or “flexible tether”) to a “leucine zipper” peptide sequence which forms a helical secondary structure. The leucine zipper helical structure dimerizes (non-covalently associates) with another identical leucine zipper sequence on another E protein subunit molecule.

The leucine zipper domain of 80E ZipperII is further modified (engineered) to contain a glycine-glycine-cysteine-glycine-glycine peptide sequence at its carboxy terminus (GGCGG sequence) which facilitates disulfide bond formation between the cysteine residues within the two leucine zipper helices. Thus, once the leucine zipper dimerizes, a disulfide bond forms between the two ends, resulting in a covalently linked dimer product. The formation of a covalently linked leucine zipper results in the dimerization of the 80E molecules, which may associate in native head to tail conformation by virtue of the flexible tether connecting the 80E molecules with the leucine zipper domain. “80E ZipperII” also refers to the corresponding peptide region of the envelope protein of other WN naturally occurring variants, as well as corresponding peptide regions of the envelope (E) protein of other Flaviviruses, for example, any serotypes of: JE, TBE, DEN, SLE and YF. WN 80E Zipper II containing a GGCGG sequence is especially preferred. The association between leucine zipper domains results in a local concentration of 80E monomers that is sufficiently high to favor the association of the conformationally correct monomers in the native quaternary head-to-tail dimeric conformation.

It would be readily apparent to one of ordinary skill in the art to select other leucine zipper sequences as well. The present invention is not limited to the specific disclosed leucine sequences, but to any amino acid sequences that would permit the dimerization with an identical sequence from another 80E molecule with flexible tether. Further, the ordinary skilled artisan would readily be able to determine other sequences that would facilitate disulfide bond formation between the two leucine zipper helices.

Another approach used to enhance dimerization of 80E is the addition of a helix-turn-helix domain to the carboxy terminal end of 80E. The helix-turn-helix domain from one modified 80E molecule will associate with that of another to form a dimeric four-helix bundle domain. Preferably, an “80E Bundle” refers to such a dimeric four-helix bundle domain and preferably to a polypeptide which encodes WN 80E-GGGSGGGGSGGGTGGGSGGGSPGEL EELLKHLKELLKGPRKGELEELLKHLKELLKGEF. The first 22 amino acids extending after the carboxy terminus of 80E serve as flexible tether between the 80E domain and the helix-turn-helix domain which follows. The formation of a non-covalently associated four-helix bundle domain will enhance the dimerization of the 80E molecules which may associate in the native head to tail conformation by virtue of the flexible tether connecting 80E to the helix bundle. “80E Bundle” also refers to the corresponding peptide region of the envelope protein of WN naturally occurring variants, as well as corresponding peptide regions of the envelope (E) protein of other Flaviviruses, for example, any serotypes of: JE, TBE, DEN, SLE and YF. The association between helix-turn-helix domains results in a local concentration of 80E monomers that is sufficiently high to favor the association of the conformationally correct monomers in the native quaternary head-to-tail dimeric conformation.

It would be readily apparent to one of ordinary skill of the art to select other amino acid sequences that would form the flexible tether extending after the carboxy terminal of the 80E and also comprising a helix-turn-helix domain. The present invention is not limited to the specific disclosed helix-turn-helix domains, but to any amino acid sequences that would enable the dimerization of one modified 80E molecule through a non-covalent association with a second modified 80E molecule. Further, the ordinary skilled artisan would readily be able to determine other sequences that would facilitate such non-covalent association of helices.

Flavivirus Non-Structural Subunits

In addition to the flavivirus envelope proteins discussed above, the immunogenic formulations of the described invention optionally include a flavivirus non-structural protein. Flavivirus non-structural (NS) proteins may include: NS1, NS2a, NS2b, NS3, NS4a, NS4b and NS5 (Chambers, supra; Henchal and Putnak, supra). In a preferred embodiment, the non-structural protein is NS1 from WN virus and is recombinantly expressed and secreted from Drosophila host cells, preferably Drosophila melanogaster Schneider (S2) cells as described in U.S. Pat. No. 6,416,763. Including a non-structural protein such as NS1 in the vaccine enhances the ability of the vaccine to elicit a cell-mediated immune response in the vaccinee, as well as an additional humoral component of immunity. Although non-structural proteins are not present in mature virions, they are produced in infected cells as a necessary part of the enzymatic system for viral replication (Mackenzie, J. M. et al., Virol., (1996) 220:232-240). Peptide epitopes processed from these proteins are displayed on the surface of infected antigen-presenting cells in association with MHC class I molecules, and thus may be recognized by a subset of immune cell populations, i.e., CD8+ T lymphocytes. When activated, this subset of immune cell populations can function as cytotoxic T cells, and thus are capable of eliminating cells infected with virus (Cane, P. A. et al., J. Gen. Virol., (1988) 69:1241-1246; Livingston, P. G., et al., J. Immunol. (1995) 154:1287-1295.; Mathew, A. et al., J. Clin. Invest. (1996) 98:1684-1692). This cellular immune response contributes to the overall protective efficacy of a subunit vaccine. Indeed, the protective efficacy of immunization with NS1 has been demonstrated for several Flaviviruses (Falgout, B. et al., J. Virol., (1990) 64(9):4356-4363; Fleeton, M. N. et al., J. Gen. Virol (1999) 80:1189-1198; Hall, R. A. et al., J. Gen. Virol., (1996) 77:1287-1294; Jacobs, S. C., et al., J. of Gen. Virol. (1994) 75:2399-2402). In addition, there is evidence that NS1 may elicit a humoral protective immune response involving the complement fixing activity of antibodies to this protein through mechanisms such as antibody-dependent, complement-mediated cytolysis, or Fc receptor mediated antibody-dependent cellular cytotoxicity (ADCC) (Schlesinger, J. J. et al., J. Immunol., (1985) 135(4):2805-2809; Schlesinger, J. J. et al., J. Virol., (1986) 60(3):1153-1155; Schlesinger, J. J., et al., J. Gen. Virol. (1987) 68:853-857; Schlesinger, J. J. et al., J. Gen. Virol. (1990) 71:593-599; Schlesinger, J. J. et al., Virology (1993) 192:132-14). Thus, the inclusion of a flavivirus non-structural protein such as NS1 in a vaccine can be justified on the basis of a humoral as well as a cellular immune response.

In a preferred embodiment, the NS1 protein produced by the Drosophila S2 cell expression system described above is also purified by IAC, but using a different monoclonal antibody (7E11), as previously described (McDonnell et al., U.S. Pat. No. 6,416,763, example 6).

Adjuvants

In addition to the antigenic components described above, the invention preferably contains an adjuvant which aids in inducing a potent, protective immune response to the conformationally relevant antigen, particularly in the immunodeficient population.

Saponin or Saponin-Based Adjuvants

In an especially preferred embodiment of the invention, a saponin or saponin-based adjuvant such as ISCOMATRIX® or GPI-0100 are added to the recombinant subunit truncated envelope protein, with or without a supplemental non-structural protein in the composition. Targeting specific antigen-presenting cell (APC) populations, listed above as one of the modes of action of adjuvants, may involve a particular receptor on the surface of the APC, which could bind the adjuvant/antigen complex and thereby result in more efficient uptake and antigen processing as discussed above. For example, a carbohydrate-specific receptor on an APC may bind the sugar moieties of a saponin such as ISCOMATRIX® or GPI-0100 (Kensil, C. R. et al., J. Immunol. (1991) 146:431-437; Newman M. J. et al., J. Immunol. (1992) 148:2357-2362; U.S. Pat. Nos.: 5,057540; 5,583,112; 6,231,859). Although the validity of the invention is not bound by this theory, a possible mechanism of action may be that if the saponin is also bound to an antigen, this antigen would thus be brought into close proximity of the APC and more readily taken up and processed. Similarly, if the adjuvant forms micellar or liposomal complexes with antigen and the adjuvant can interact or fuse with the APC membrane, this may allow the antigen access to the cytosolic or endogenous pathway of antigen processing. As a result, peptide epitopes of the antigen may be presented in the context of MHC class I molecules on the APC, thereby inducing the generation of CD8+ cytotoxic T lymphocytes (“CTL”; Newman et al., supra; Oxenius, A., et al., J. Virol. (1999) 73: 4120).

A saponin is any plant glycoside with soapy action that can be digested to yield a sugar and a sapogenin aglycone. Sapogenin is the nonsugar portion of a saponin. It is usually obtained by hydrolysis, and it has either a complex terpenoid or a steroid structure that forms a practicable starting point in the synthesis of steroid hormones. The saponins of the invention can be any saponin as described above or saponin-like derivative with hydrophobic regions, especially the strongly polar saponins, primarily the polar triterpensaponins such as the polar acidic bisdesmosides, e.g. saponin extract from Quillsjabark Araloside A, Chikosetsusaponin IV, Calendula-Glycoside C, chikosetsusaponin V, Achyranthes-Saponin B. Calendula-Glycoside A, Araloside B, Araloside C, Putranjia-Saponin III, Bersamasaponiside, Putrajia-Saponin IV, Trichoside A, Trichoside B, Saponaside A, Trichoside C, Gypsoside. Nutanoside, Dianthoside C, Saponaside D, aescine from Aesculus hippocastanum or sapoalbin from Gyposophilla struthium, preferably, saponin extract Quillaja saponaria Molina and Quil A. In addition, saponin may include glycosylated triterpenoid saponins derived from Quillaja Saponaria Molina of Beta Amytin type with 8-11 carbohydrate moieties as described in U.S. Pat. No. 5,679,354. Saponins as defined herein include saponins that may be combined with other materials, such as in an immune stimulating complex (“ISCOM”)-like structure as described in U.S. Pat. No. 5,679,354. Saponins also include saponin-like molecules derived from any of the above structures, such as GPI-0100, such as described in U.S. Pat. No. 6,262,029.

Preferably, the saponins of the invention are amphiphilic natural products derived from the bark of the tree, Quillaia saponaria. Preferably, they consist of mixtures of triterpene glycosides with an average molecular weight (Mw) of 2000. A particularly preferred embodiment of the invention is a purified fraction of this mixture.

The most preferred embodiment of the invention is WN 80E combined with ISCOMATRIX® GPI-0100 to produce a vaccine formulation able to induce potent, safe, protective immune responses in vaccinated subjects, including members of the immunodeficient population.

Emulsion and Emulsion-Based Adjuvants

In another preferred embodiment, an emulsion or emulsion-based adjuvant, such as Co-Vaccine HT, is added to the recombinant subunit E protein, with or without a non-structural protein in the composition. Emulsions and emulsion-based vaccines are known in the art (Podda A. and G. DelGiudice, Expert Rev. Vaccines (2003) 2:197-203; Banzhoff A. et al., Gerontology (2003) 49:177-84) and are believed to function primarily through a depot effect, although CoVaccine HT does not function in this manner. Rather it most likely functions as an immune modulator, since it contains carbohydrate moieties bound to micro-droplets of vegetable oil, which is thought to mimic the bacterial cell surface. Thus, while this adjuvant is physically an emulsion, its mode of action as an adjuvant is that of an immunomodulator.

While reports in the literature (Podda and DelGiudice supra; Banzhoff et al., supra) have suggested that addition of emulsions such as MF59 to subunit vaccines may overcome some of the immune difficulties in the elderly, side by side evaluation with another unadjuvanted, subunit-like (split influenza) vaccine formulation failed to show an added benefit (Ruf et al., supra), resulting in a lack of medical endorsement for the use of the MF59 adjuvanted vaccine (Prescrire Int. supra). Addition of carbohydrates or immunostimulatory molecules to an emulsion (e.g., Co-Vaccine HT) is believed to further enhance the adjuvant effect through more effective stimulation of antigen presenting cells as described above. Thus, the combination of an emulsion-based adjuvant containing additional immunostimulatory molecules with a conformationally relevant recombinant flavivirus envelope protein produces a particularly potent vaccine composition. In a highly preferred embodiment, WN 80E is combined with Co-Vaccine HT to produce a vaccine formulation able to induce potent, protective immune responses in vaccinated subjects, including members of the immunodeficient population.

Alum

In a preferred embodiment, the recombinant subunit truncated flavivirus E protein, with or without non-structural proteins in the composition, is formulated with aluminum-based adjuvants (collectively, “alum” or “alum-based adjuvants”) such as aluminum hydroxide, aluminum phosphate, or a mixture thereof. Aluminum hydroxide (commercially available as “Alhydrogel”) was used as alum in the Examples. Aluminum-based adjuvants remain the only adjuvants currently registered for human use in the United States and their effectiveness is widely recognized. Alum-based adjuvants are believed to function via a depot mechanism and the combination of the conformationally relevant flavivirus envelope antigen with the depot effect is sufficient to induce a potent immune response in vaccinated individuals, including members of the immunodeficient population.

Oligodeoxyribonucleotide

Synthetic oligodeoxyribonucleotides (ODNs) containing unmethylated cytosine-guanosine dinucleotides (CpG-ODNs) stimulate immune system cells. Optimally active K-type ODNs have a phosphorothioate backbone and express multiple unmethylated CpG dinucleotides flanked by a 5′ thymidine (T) and a TpT or ApT dinucleotide at the 3′-flanking position. D-type ODNs are structurally complex. Optimally active D-type ODNs contain a central purine/pyrimidine/CpG/purine/pyrimidine motif flanked on both sides by 3-4 self-complementary bases. (See Verthelyi & Klinman, Clinical Immunology, (2003) 109:64-71).

In vitro, CpG-ODNs directly activate B cells and plasmacytoid dendritic cells. CpG-ODNs have also been reported to indirectly activate monocytes, macrophages, NK cells, and memory T cells. In vivo, CpG-ODNs have been reported to be potent adjuvants that promote cellular and humoral immune responses. For example, particularly encouraging results have been reported in a study of an oligonucleotide adjuvant with a recombinant subunit viral vaccine (hepatitis B vaccine) in humans. The reported combination showed that the adjuvant enhanced the immune response to the vaccine, while being well-tolerated, both locally and systemically. Those of ordinary skill in the art will recognize, however, that the efficacy of any given adjuvant is immunogen dependent and thus predicting which combinations will be successful is difficult.

In a preferred embodiment, an immunostimulatory oligonucleotide is synthetic, between 2 to 100 base pairs in size and contains a consensus mitogenic CpG motif represented by the formula:
5′ X₁X₂CG X₃ X₄ 3′
wherein C and G are unmethylated, X₁, X₂, X₃ and X₄ are nucleotides and a GCG trinucleotide sequence is not present at or near the 5′ and 3′ termini. (See U.S. Pat. No. 6,194,388, which is hereby incorporated by reference in its entirety.)

Preferably, oligodeoxyribonucleotides (ODNs) for use with the disclosed invention are in the range of about 20-24 nucleotides length, although ODN sequences with as few as 6 nucleotides have been reported to be effective also (Wang, S. et al, Vaccine (2003) 21:4297-4306). Each one contains a “CpG” sequence in the middle of the ODN. These “CpG” dinucleotide sequences are unmethylated, thus mimicking those nucleotides found in bacterial DNA, in contrast to vertebrate DNA, in which the CpG sequences are methylated (and underrepresented, i.e., suppressed).

Some examples of ODNs are listed below:

1) CpG ODN 1826: TCCATGACGTTCCTGACGTT;
2) CpG ODN 1760: ATAATCGACGTTCAAGCAAG;
3) non-CpG ODN 1908: ATAATAGAGCTTCAAGCAAG;
4) non-CpG ODN 1745: TCCAATGAGCTTCCTGAGTCT;
5) hexamer CpG: GACGTT;
6) D-ODN D35: GGTGCATCGATGCAGGGGGG;
7) D-ODN 2216: GGTGCATCGATGCAGGGGGG;
8) K-ODN DSP3O: TCGTCGCTGTCTCCGCTTCTTCTTGCC;
9) K-ODN 2006: TCGTCGTTTTGTCGTTTTGTCGTT;
10) K-ODN K3: ATCGACTCTCGAGCGTTCTC;
11) K-ODN K23: TCGAGCGTTCTC;
and
12) K-ODN ISS: TGACTGTGAACGTTCGAGATGA.

(A = adenosine, C = cytidine, G = guanosine, T = thymidine).

In an alternative embodiment, cytosine-guanosine-independent ODNs (non-CpG ODNs) may be used as adjuvants with the disclosed methods. Non-CpG ODNs typically comprise the general formula PyNTTTTGT in which Py is C or T, and N is A, T, C, or G. (Elias, et al., J. Immun. (2003) 171:3697-3704.) Non-CpG ODNs may be used alone or with other adjuvants and may also be used with CpG ODNs.

Diagnostics

In a preferred embodiment, the recombinant flavivirus 80E antigens may be used as analytical reagents for assessing the presence or absence of anti-flavivirus antibodies in samples. The antigens may be used in standard immunoassay formats with standard detection systems such as enzyme-based (ELISA), fluorescence-based, or isotope-based detection systems. Preferably, the antigen is coupled or adsorbed to a solid support or in sandwich format, but a multiplicity of protocols are possible and standard in the art. In a most preferred embodiment WN 80E is used as an analytical reagent for assessing the presence or absence of anti-WN antibodies in samples.

In another preferred embodiment, the recombinant flavivirus 80E antigens are used to assess the quality of the antibody response through measurement of binding affinity or avidity. Various methods exist in the art, including addition of chaotropic agents or use of plasma resonance platforms (e.g., Biacore).

Production of Anti-Flaviviral Immune Cells or Antibodies

In a preferred embodiment, the recombinant flavivirus 80E or NS1 antigens may be used as immunogens to produce transformed immune B cells or hybridomas following immunization of subjects with said antigens. Transformed immune B cells or hybridomas produced in such a manner may be used to produce polyclonal or monoclonal antibody preparations which are reactive with the recombinant antigen and may be used as reagents for ill vitro testing, or passive immunotherapy in either a prophylactic or therapeutic setting. Immune B cells and polyclonal antisera can be generated upon immunization of subjects and sampling of peripheral blood according to standard methods recognized in the art. Similarly methods for producing transformed B cells, hybridomas, and monoclonal antibodies are known in the art. In a highly preferred embodiment, monoclonal or polyclonal antibodies produced following immunization with the recombinant WN 80E are used in passive immunotherapy of exposed or potentially exposed individuals.

Identification and Screening of Antiviral compounds

In a preferred embodiment, the recombinant flavivirus 80E or NS1 proteins may be used to identify and/or screen for antiviral compounds which could be effective for preventing or limiting disease induced by the infecting flavivirus. In a particularly preferred embodiment, the crystal structure of WN 80E is used for identification of regions which are possible targets for small molecule anti-West Nile virus development and subsequently used for screening candidate compounds.

Administration and Use

The described invention thus concerns and provides a means for preventing or attenuating infection by Flavivirus. As used herein, a vaccine is said to prevent or attenuate a disease if administration of the vaccine to an individual results either in the total or partial immunity of the individual to the disease, or in the total or partial attenuation (i.e., suppression) of a symptom or condition of the disease.

A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient.

The active vaccines of the invention can be used alone or in combination with other active vaccines such as those containing other active subunits to the extent that they become available. Corresponding or different subunits from one or several serotypes may be included in a particular formulation.

The therapeutic compositions of the described invention can be administered parenterally by subcutaneous, intramuscular, or intradermal injection.

Many different techniques exist for the timing of the immunizations when a multiple administration regimen is utilized. It is preferable to use the compositions of the invention more than once to increase the levels and diversities of expression of the immunoglobulin repertoire expressed by the immunized subject. Typically, if multiple immunizations are given, they will be given one to two months apart.

To immunize subjects against WN-induced disease for example, the vaccines containing the subunit(s) are administered to the subject in conventional immunization protocols involving, usually, multiple administrations of the vaccine. Administration is typically by injection, typically intramuscular or subcutaneous injection; however, other systemic modes of administration may also be employed.

According to the described invention, an “effective amount” of a therapeutic composition is one which is sufficient to achieve a desired biological effect. Generally, the dosage needed to provide an effective amount of the composition will vary depending upon such factors as the subject's age, condition, sex, and extent of disease, if any, and other variables which can be adjusted by one of ordinary skill in the art. The antigenic preparations of the invention can be administered by either single or multiple dosages of an effective amount. Effective amounts of the compositions of the invention can vary from 0.01-100 μg per dose, more preferably from 0.1-20 μg per dose, and most preferably 1-5 μg per dose.

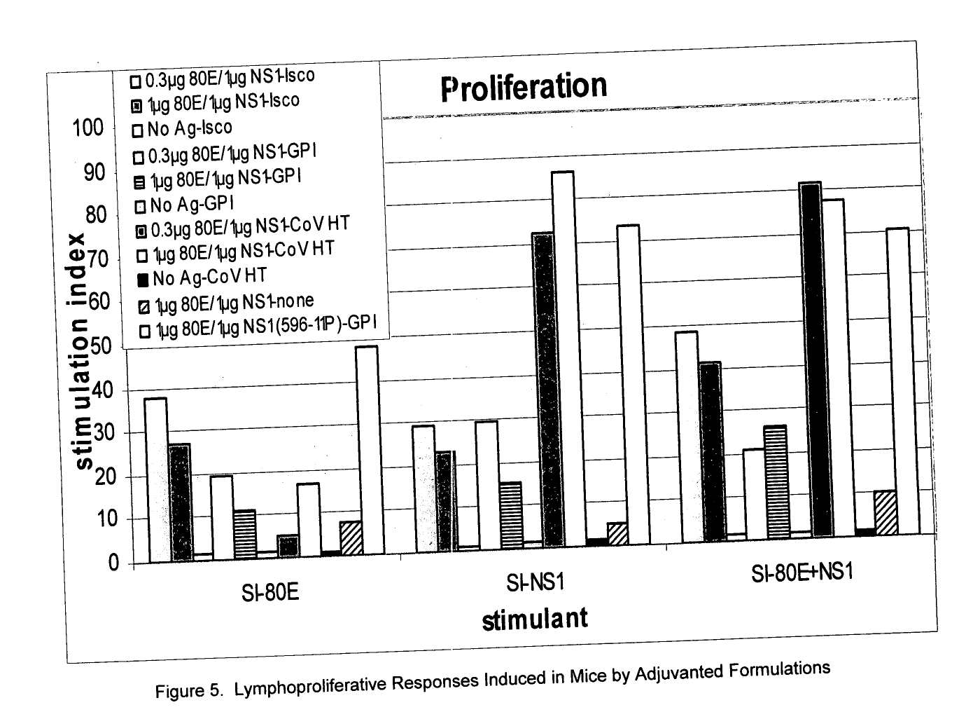

The Examples below demonstrate the ability of selected candidate West Nile vaccine formulations to induce potent and protective immune responses in vaccinated individuals. The immunogenicity and efficacy of the selected formulations depend on the novel combination of two different aspects. In one aspect the production of conformationally relevant recombinant WN 80E antigen in quantities sufficient to be of practical use is disclosed in the invention (Examples 1 and 2). In a second aspect, the combination of the relevant 80E antigen with particular adjuvants shown through experimentation to enhance the immunogenicity of the WN subunit vaccine is disclosed (Examples 3-20). The unique combination of these aspects results in the novel invention of highly immunogenic WN vaccine formulations which induce high titer virus neutralizing responses in numerous species. Furthermore, in addition to the antibody responses reported in Tables 2, 3, 5, and 8, the Examples demonstrate that the WN recombinant subunit vaccines formulated with the adjuvant ISCOMATRIX®, GPI-0100, or Co-Vaccine HT elicit a robust cell-mediated (“Th1” type) immune response (in addition to a “Th2” response) as indicated by lymphocyte proliferation and antigen-stimulated production of high levels of IFN and IL-5 from immune splenocytes in vitro (FIGS. 5, 6, and 7). These unique combinations of conformationally correct WN antigen and selected adjuvants which facilitate induction of strong immune responses, including the Th1 type, are uniquely situated to address the technical problem of inducing relevant protective immune responses in vaccinated individuals, including overcoming the immune limitations in the elderly and other members of the immunodeficient population, while maintaining an acceptable safety profile.

The following examples are intended to illustrate but not to limit the invention.

### EXAMPLE 1

### Expression of West Nile 80E and NS1 Proteins in the Drosophila S2 System

The expression plasmid pMttbns (derived from pMttPA) contains the following elements: Drosophila melanogaster metallothionein promoter, the human tissue plasminogen activator secretion leader (tPAL) and the SV40 early polyadenylation signal. At Hawaii Biotech, a 14 base pair BamHI (restriction enzyme from Bacillus amyloliqufacience) fragment was excised from the pMttbns vector to yield pMttΔXho that contains a unique XhoI (restriction enzyme from Xanthomonas holicicola) site in addition to an existing unique BglII (restriction enzyme from Bacillus globigii) site. This expression vector promotes the secretion of expressed proteins into the culture medium. All West Nile sequences were introduced into the pMttΔXho vector using these unique BglII and XhoI sites. For the expression of a carboxy-truncated West Nile envelope protein, a synthetic gene encoding the prM protein and 80% of the E protein from West Nile virus was synthesized (Midland Certified Reagent Co., Midland, Tex.). The nucleotide sequence of the synthetic gene follows the published sequences of West Nile viruses isolated in 1999 in New York City. The C-terminal truncation of the E protein at amino acid 401 eliminates the transmembrane domain of the E protein (in a fashion analogous to Hawaii Biotech's dengue envelope protein vaccines), and therefore can be secreted into the medium. For the expression of a full-length West Nile NS1 protein a gene fragment was generated by RT-PCR. The NS1 gene fragment represents nucleotides 2470 to 3525 on the genome and codes for a product containing 352 amino acid residues. Both the synthetic prM80E (pre-membrane protein-80% glycoprotein E) gene fragment and RT-PCR (reverse transcriptase-polymerase chain reaction) generated NS1 gene fragment include restriction endonuclease sites that were used for cloning and also included two stop codons immediately following the last West Nile codon. The final prM80E plasmid construct was designated pMttWNprM80E and the NS1 plasmid construct was designated pMttWNNS1.

S2 cells were co-transformed with both the pMttΔXho-based expression plasmids and the pCoHygro selection plasmid that encodes hygromycin resistance utilizing the (i) calcium phosphate co-precipitation method or (ii) Cellfectin (Invitrogen Kits, Carlsbad, Calif.) according to the manufacturer's recommendations. Cells were co-transformed with 20 μg total DNA with a 20:1 ratio of expression plasmid to selection plasmid. Transformants were selected with hygromycin B (Roche Molecular Biochemicals, Indianapolis, Ind.) at 300 μg/ml. Following selection, cells were adapted to growth in the serum free medium, Excel 420 (JRH, Lenexa, Kans.). For expression studies, cells were grown in Excel 420, 300 μg/ml hygromycin, and induced with 200 μM CuSO4. Cells were seeded at a density of 2×10⁶ cells/ml and allowed to grow for 6-7 days. Under optimal conditions, cell densities of 1.5 to 2×10⁷ cells/ml were achieved after 6-7 days of growth. The culture supernatant was examined for expressed protein by SDS-PAGE and Western blot.

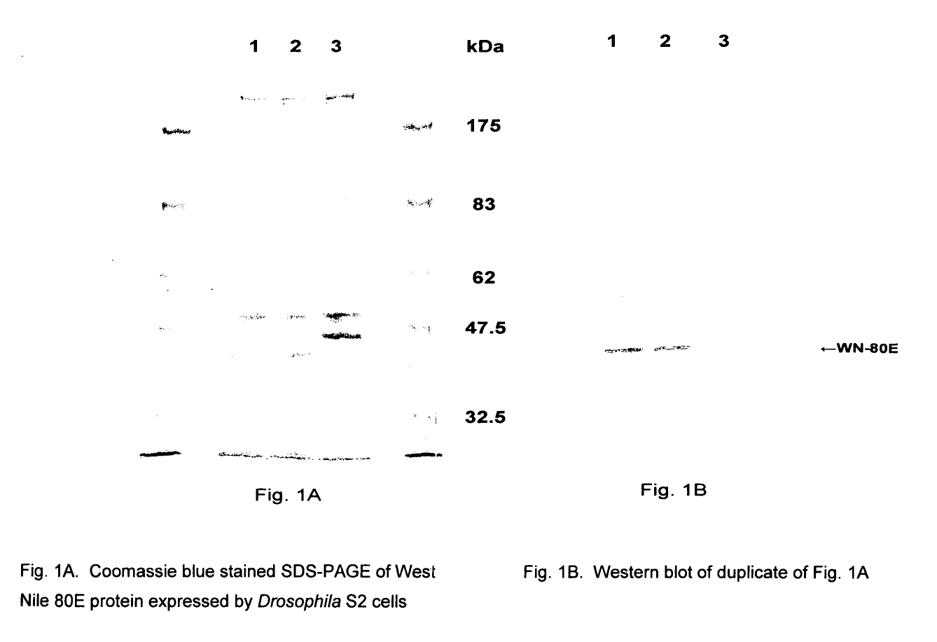

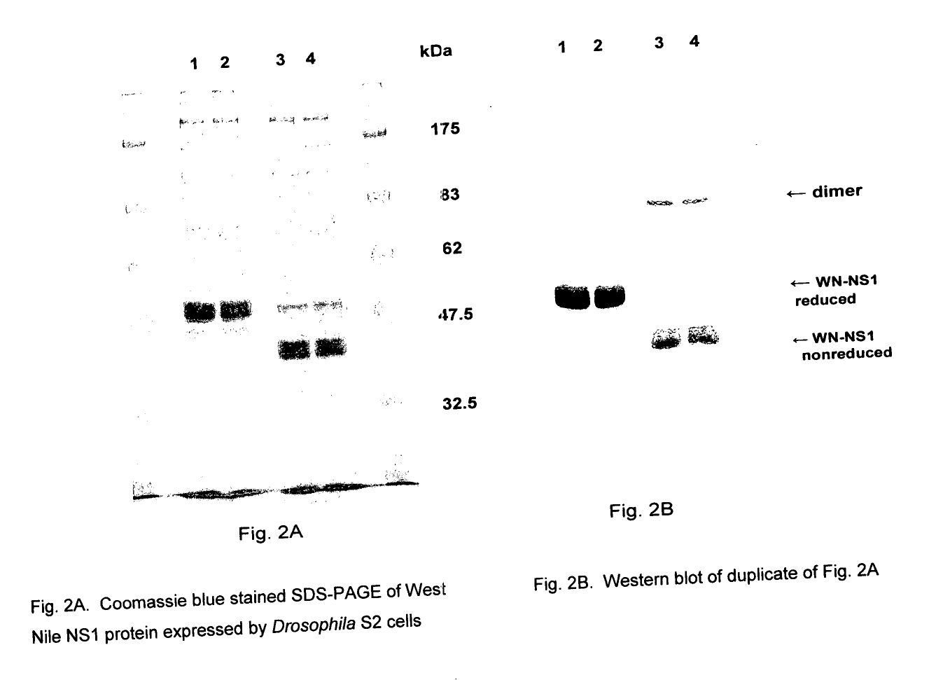

For the detection of West Nile 80E on Western blots a rabbit polyclonal anti-West Nile virus antibody (BioReliance Corp., Rockville, Md.) followed by an anti-rabbit IgG-alkaline phosphatase conjugated secondary antibody was used. For the detection of West Nile NS1 on Western blots the flavivirus group specific anti-NS1 monoclonal 7 E11 followed by an anti-mouse IgG-alkaline phosphatase conjugated secondary antibody was used. The blots were developed with NBT/BCIP (Sigma Chem. Co.) solid phase alkaline phosphatase substrate. Results are shown in FIGS. 1A and 1B and 2A and 2B.

### EXAMPLE 2

### Purification of West Nile 80E and NS1

Purification protocols were developed for both the West Nile subunit envelope protein (80E) and non-structural protein 1 (NS1). The procedures are based upon existing methods that are currently utilized for manufacturing of dengue antigens for in vitro diagnostic use and intended to be utilized for the manufacture of a dengue vaccine. Purification of both proteins was accomplished by immunoaffinity chromatography (IAC). For 80E, the monoclonal antibody (MAb) 4G2 was utilized, while the monoclonal antibody 7E11 was utilized for purification of NS1. Briefly, the procedure involves filtration of the post-expresssion medium using a Whatman 1 filter. The crude material is then loaded onto the IAC column, which contains immobilized MAb that is covalently coupled via N-hydroxysuccinimide chemistry, at a linear flowrate of 2 cm/min for 80E and 1.2 cm/min for NS1. After the sample is loaded, the matrix is washed with 10 mM phosphate buffered saline (PBS), pH 7.2, containing 0.05% (v/v) tween-20 (PBST, 140 mM NaCl). Bound protein is eluted from the IAC column with 20 mM glycine buffer, pH 2.5. The eluate is neutralized then buffer exchanged against PBS with or without tween (for 80E) or 10 mM phosphate buffer (for NS1). The purification products are routinely analyzed by sodium dodecyl sulfate polyacrylamide gel electrophoresis (SDS-PAGE) with Coomassie or silver staining, Western blot, UV absorption, and sandwich ELISA to determine purity, identity, quantity, and bioactivity, respectively. In addition, samples were analyzed by N-terminal amino acid sequencing and amino acid analysis. These analyses provided confirmation of identity and quantity of the purification products. Yields from the columns have proven to be consistent for both proteins with satisfactory recoveries, thus indicating that, if used in the current formats, these processes should be suitable for production volumes required for product manufacturing.

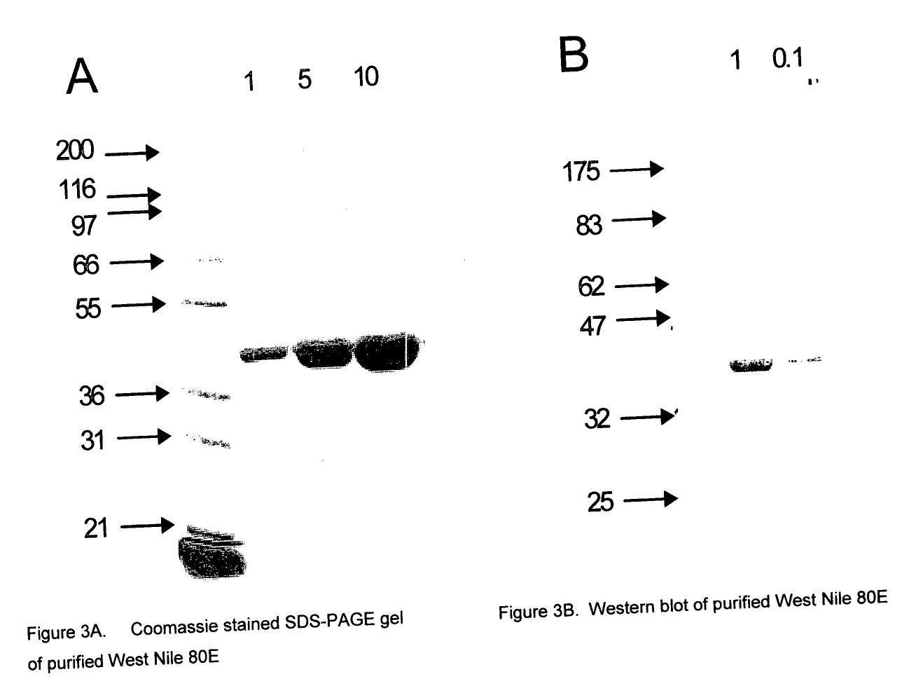

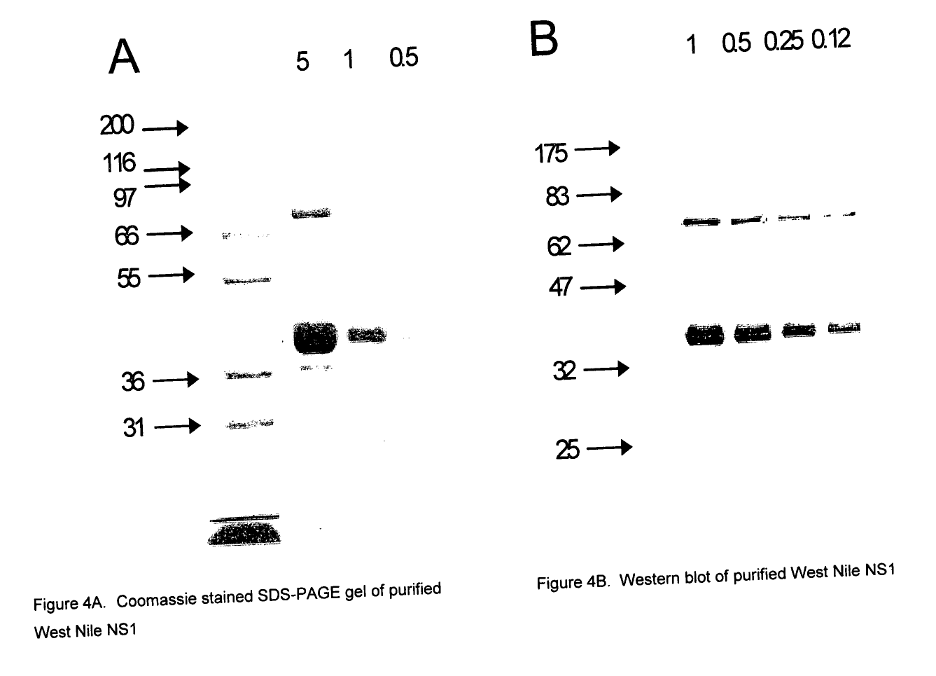

Representative SDS-PAGE and Western blot profiles of the two purified proteins are presented in FIGS. 3 and 4. For the analysis, both samples were run under non-reducing conditions. The 80E molecule migrates as a single band with a relative molecular weight consistent with that determined from the amino acid composition (i.e., 43 kD). This finding indicates that disulfide bond formation is not occurring between molecules, although aggregates (e.g. dimers) stabilized by noncovalent interactions could still be present in the native state. Trace contaminants (˜5 bands) are visible in a 10 μg load on the Coomassie stained gel. Assuming a threshold of detection of 100 ng, the purity of the 80E can be estimated at >90%. In contrast, the NS1 migrates as two distinct forms: one with a relative molecular weight that is consistent with that expected for a monomeric form (40 kD) and one with a relative molecular weight that is consistent with a dimeric form (80 kD). Unlike the 80E preparation, a major contaminant is clearly visible in a 5 μg load with possibly 2-3 minor contaminants as well. As the major contaminant is still visible in a 1 μg load but not a 0.5 μg load, the purity of the NS1 preparations are estimated at ˜90%.

### EXAMPLES 3-6

### Immune Response to Adjuvanted West Nile Vaccine Formulations in Mice

### EXAMPLE 3 Vaccines formulated with ISCOMATRIX® in Balb/c mice

Balb/c mice (8 weeks old) were vaccinated twice, subcutaneously, with a 4 week interval with the indicated amounts (see below) of 80E and NS1 antigens plus the saponin-based adjuvant ISCOMATRIX®. The antibody response to vaccination was determined on serum samples collected from individual mice 14 days post booster vaccination. Sera were titrated for antibodies to both the 80E and NS1 proteins by a standard ELISA technique using plates coated with the individual antigens. Alternatively, the ability to neutralize live virus in vitro was assessed using a plaque reduction neutralization test (PRNT). Dilutions of antisera are incubated with a defined amount of West Nile virus (Egypt 101 strain) then plated onto Vero cell monolayers. The number of resulting plaques are compared with the number present in the no serum virus control and the percent reduction calculated. The highest dilution of serum which results in at least 90% reduction in plaque counts is considered the PRNT₉₀ titer. The results of these assays given in Table 2 demonstrate that the vaccine elicits a high titer antibody response in mice to 80E at doses of 1-10 μg and there was no negative effect on addition of NS1 to the vaccine formulation.

TABLE 2
PRNT₉₀^{a} Virus Neutralization Titers in Mice Immunized with West Nile
80E +/− NS1 in combination with ISCOMATRIX ® Adjuvant

| Group | | | | PRNT |
|---|---|---|---|---|
| Number | 80E dose (μg) | NS1 dose (μg) | Adjuvant^{b} | Titer^{a} |
| 1 | 1 | 0 | ISCOMATRIX ® | 1:5120 |
| 2 | 1 | 0.3 | ISCOMATRIX ® | NT |
| 3 | 1 | 1 | ISCOMATRIX ® | 1:5120 |
| 4 | 3 | 0 | ISCOMATRIX ® | 1:1280 |
| 5 | 3 | 0.3 | ISCOMATRIX ® | NT |
| 6 | 3 | 1 | ISCOMATRIX ® | 1:5120 |
| 7 | 10 | 0 | ISCOMATRIX ® | 1:5120 |
| 8 | 10 | 0.3 | ISCOMATRIX ® | NT |
| 9 | 10 | 1 | ISCOMATRIX ® | 1:5120 |
| 10 | 0 | 0 | ISCOMATRIX ® | <1:10 |

^{a}PRNT₉₀ titer: highest dilution of serum yielding ≧90% reduction in the number of plaques compared to virus controls. NT: not tested
^{b}dose = 5 μg

### EXAMPLE 4

### Immunogenicity of West Nile Antigens in Combination with GPI-0100 Adjuvant in Balb/c Mice

Groups of Balb/c mice each were immunized subcutaneously with two doses of the West Nile 80E antigen with and without NS1 in combination with 100 μg of the saponin-based GPI-0100 adjuvant at a 4 week interval. Various doses of 80E and NS1 were examined in this study. Virus neutralization responses were assessed as described in Example 3. Details of the groups are included in Table 3 below. All doses of 80E induced high titer virus neutralizing responses.

TABLE 3
West Nile Virus Neutralizing Antibody Responses Induced by Candidate
Adjuvanted Recombinant Subunit in Mice

| Group no. | 80E dose (μg) | NS1 dose (μg) | Adjuvant^{a} | PRNT Titer |
|---|---|---|---|---|
| 1 | 0.3 | 0 | GPI-0100 | 1:1280 |
| 2 | 0.3 | 1 | GPI-0100 | 1:1280 |
| 3 | 1 | 0 | GPI-0100 | 1:5120 |
| 4 | 1 | 1 | GPI-0100 | 1:1280 |
| 5 | 0 | 0 | GPI-0100 | <1:10 |

^{a}GPI-0100 (Australian Cancer Technology, Birmingham, Alabama), 100 μg

### EXAMPLE 5

### Immunogenicity of West Nile 80E+NS1 in Combination with Saponin and Emulsion-Based Adjuvants in Swiss Webster Mice

Groups of 10 female Swiss Webster mice were immunized twice subcutaneously at a 4 week interval with 80E at various doses plus NS1 in combination with several adjuvants as detailed in Table 4 below. Serum was obtained approximately 2 weeks after the second dose and immune responses assessed as described in Example 3.

TABLE 4
Groups for Adjuvant Comparison Study in Mice

| Group | 80E Dose (μg) | NS1 Dose (μg) | Adjuvant |
|---|---|---|---|
| 1 | 0.3 | 1.0 | ISCOMATRIX ®^{a} |
| 2 | 1.0 | 1.0 | ISCOMATRIX ® |
| 3 | 0 | 0 | ISCOMATRIX ® |
| 4 | 0.3 | 1.0 | GPI-0100^{b} |
| 5 | 1.0 | 1.0 | GPI-0100 |
| 6 | 0 | 0 | GPI-0100 |
| 7 | 0.3 | 1.0 | CoVaccine HT^{c} |
| 8 | 1.0 | 1.0 | CoVaccine HT |
| 9 | 0 | 0 | CoVaccine HT |
| 10 | 1.0 | 1.0 | None |
| 11 | 1.0^{d} | 1.0 | GPI-0100 |

^{a}12 μg per dose
^{b}250 μg per dose
^{c}50 μl per dose
^{d}different lot of 80E compared to other groups

Humoral immune responses were assessed as described in Example 3 or with classical hemagglutination inhibition assays and are summarized in Table 5 below. All adjuvanted formulations induced high titered virus neutralizing responses as measured by PRNT assay as well as hemagglutination inhibiting antibodies (Tesh, R. B. et al., Emerg. Inf. Dis. (2002) 8:1392) and antigen binding antibodies as measured by ELISA. In contrast the unadjuvanted formulation was less potent at inducing humoral responses.

TABLE 5
Humoral Immune Responses Induced by Adjuvanted and Unadjuvanted
Formulations in Mice

| Group | HI | PRNT₉₀ | 80E ELISA | NS1 ELISA |
|---|---|---|---|---|
| 1 | 320 | 640 | 530 | 7090 |
| 2 | 1280 | 1280 | 44,100 | 4720 |
| 3 | <20 | <10 | <250 | <100 |
| 4 | 320 | 640 | 250 | 9540 |
| 5 | 1280 | 640 | 8800 | 5390 |
| 6 | <20 | <10 | <250 | <100 |
| 7 | 1280 | 1280 | 8360 | 10,640 |
| 8 | 1280 | 2560 | 18,000 | 7120 |
| 9 | <20 | <10 | <250 | <100 |
| 10 | 20 | pending | <250 | 260 |
| 11 | 1280 | 2560 | 9260 | 4790 |

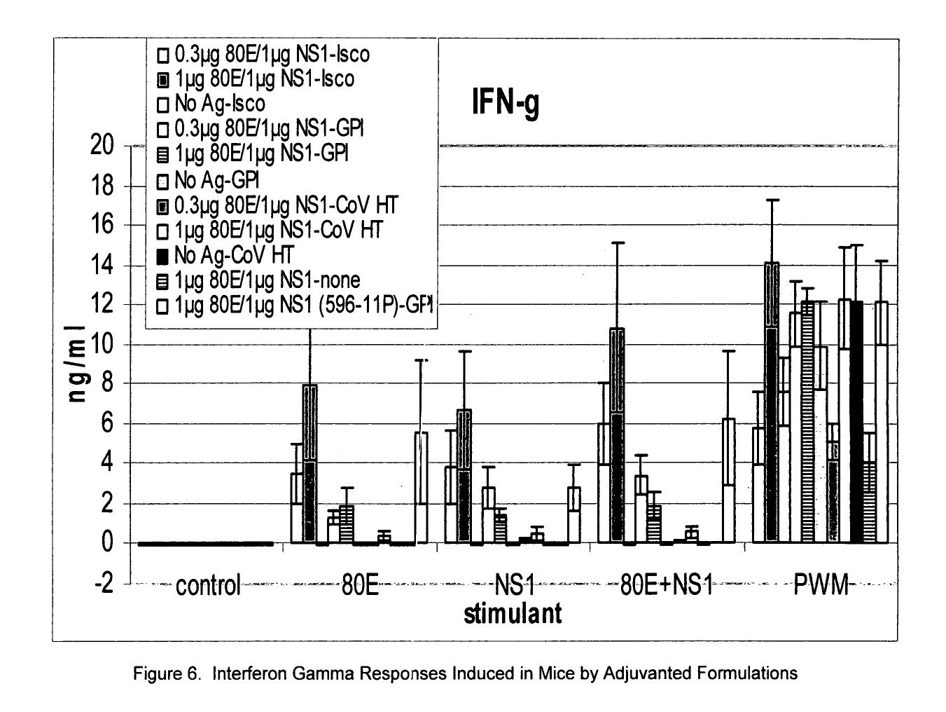

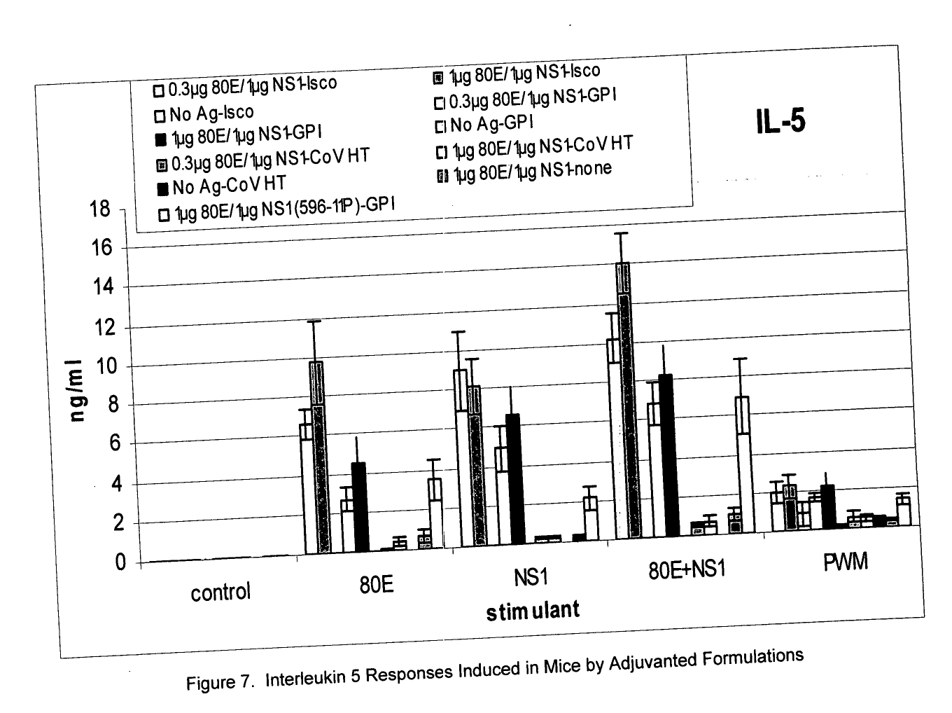

Cell mediated immune responses were also assessed as follows. Seven days post booster vaccination, splenectomies were performed on 5 mice from each group and splenocyte suspension prepared. Erythrocytes were lysed with an NH₄Cl based lysis solution, and the cell pellet resuspended in cell culture medium. Cell counts were performed on each suspension using a hemacytometer, and the suspensions diluted to 4×10⁶ cells/ml for lymphocyte proliferation and cytokine production assays. Aliquots (0.1 ml) of each splenocyte suspension were dispensed into wells of a 96-well cell culture plate. Aliquots (0.1 ml) of the West Nile antigens (80E and/or NS1) were then dispensed into the wells containing each of the cell suspensions (in quadruplicate), at a final concentration of 5 μg/ml of each antigen. Wells with unstimulated (antigen omitted) cell suspensions, as well as phytohemagglutinin (PHA) stimulated cell suspensions (as a positive control) were also included. Cultures were incubated at 37° C./5% CO₂/humidified for 7 days (3 days for PHA stimulated cultures), and then one microcurie of tritiated (methyl-3H) thymidine (60 Ci/mmol; ICN Biomedicals, Inc., Irvine, Calif.) was added to each well (in a volume of 0.01 ml), and incubation continued for 18 hrs. After that period of time, the cell cultures were harvested onto glass fiber filtration plates and washed extensively using a vacuum driven harvester system (Filtermate Plate Harvester, Perkin-Elmer Co., Boston, Mass.). The filtration plates were then analyzed for radioactivity using the TopCount Microplate Scintillation and Luminescence Counter (Perkin-Elmer Co., Boston, Mass.). Aliquots (0.5 ml) of each splenocyte suspension were also dispensed into wells of a 24-well cell culture plate. Aliquots (0.5 ml) of the same antigens used for lymphocyte proliferation were dispensed into the wells containing each of the cell suspensions. Unstimulated and pokeweed mitogen (PWM)-stimulated cell suspensions were also included. Cultures were incubated for 5 days at 37° C./5% CO₂/humidified. The culture supernatants were then harvested and frozen prior to analysis for specific cytokines. The cytokines interferon-gamma (IFN-γ) and interleukin-5 (IL-5) were assayed by a flow cytometric bead array assay. Strong lymphoproliferative responses (stimulation index >3 but often in the range of 15-30) were induced by the adjuvanted protein formulations as shown in FIG. 5. Stimulation in vitro with either 80E, NS1, or a pool of both antigens yielded similar results. Cytokine secretion was also assessed following in vitro stimulation with individual or pooled antigens. Strongest IFN-_{γ} and IL-5 responses were induced by formulations containing the saponin based adjuvants ISCOMATRIX® or GPI-0100 (FIGS. 6 and 7).

### EXAMPLE 6

### Immunogenicity of West Nile 80E in Combination with Various Adjuvants in Mice

Groups of 10 female Swiss Webster mice were immunized twice subcutaneously at a 4 week interval with 80E or mock antigen (prepared from mock transformed Drosophila media in a manner similar to the 80E antigen) in combination with several adjuvants as detailed in Table 6 below. Serum was obtained approximately 2 weeks after the second dose and virus neutralization responses assessed as described in Example 3.

TABLE 6
Groups for Adjuvant Comparison Study in Mice

| Group | Antigen | Adjuvant |
|---|---|---|
| 1 | 1 μg 80E | ISCOMATRIX ®^{a} |
| 2 | 1 μg 80E | GPI-0100^{b} |
| 3 | 1 μg 80E | GPI-0100^{c} |
| 4 | 1 μg 80E | Alum^{d} |
| 5 | 1 μg 80E | None |
| 6 | 1 μg mock | ISCOMATRIX ®^{a} |
| 7 | 1 μg mock | GPI-0100^{b} |
| 8 | 1 μg mock | GPI-0100^{c} |
| 9 | 1 μg mock | Alum^{d} |
| 10 | 1 μg mock | None |

^{a}12 μg dose
^{b}100 μg dose
^{c}250 μg dose
^{d}3 mg dose

### EXAMPLE 7

### Protective Efficacy of West Nile Vaccine Formulations in the Golden Hamster Model

The protective efficacy and immunogenicity of the West Nile 80E+/−NS1 vaccine candidates were evaluated in the golden hamster model of West Nile encephalitis (Xiao, S—Y et al., Emerg. Infect. Dis. 7:714-721, 2001; Tesh, R. B. et al., Emerg. Infect. Dis. 8:245-251, 2002). Female golden hamsters(15 per group) were immunized, subcutaneously, with the individual vaccine formulations 80E, NS1, or 80E+NS1 in combination with 12 μg ISCOMATRIX® adjuvant as detailed in Table 7 below. The control group of 15 hamsters was administered adjuvant and mock antigen only. Hamsters were given a second immunization approximately 4 weeks post dose 1. Approximately 2 weeks after the second vaccination, hamsters from each group were bled and antibody titers to West Nile virus determined by hemagglutination inhibition (HI), complement fixation (CF), and PRNT assays as described (Tesh, R. B. et al., supra). Immediately after the blood samples were obtained, all hamsters were challenged by administration of 10⁴TCID₅₀(50% tissue culture infective dose) of live virus (West Nile virus strain NY 385-99). Six randomly selected hamsters from each group were bled daily for 6 days following challenge to determine the level of viremia and the antibody response to viral challenge. Animals were held for 30 days following challenge for observation of morbidity and mortality. At the end of the 30 day holding period, the surviving animals were bled once more for antibody determinations, and then euthanized.

TABLE 7
Definition of Groups for Golden Hamster Challenge Study

| Group | 80E Dose (μg) | NS1 Dose(μg) |
|---|---|---|
| 1 | 0 | 0 |
| 2 | 0 | 1 |
| 3 | 1 | 0 |
| 4 | 1 | 1 |
| 5 | 10 | 0 |
| 6 | 10 | 1 |

Results of the analysis of humoral immune responses post dose 2 of vaccine are summarized in Table 8. Formulations containing 80E induced HI and PRNT titers while formulations containing 80E or NS1 induced CF titers.

TABLE 8
Humoral Immune Responses Induced by ISCOMATRIX ® Adjuvanted
80E and NS1 Subunit Formulations in Golden Hamsters

| | Antibody Titers^{a} | | |
|---|---|---|---|
| Group | HI | CF | PRNT₉₀ |
| 1 | <20 | <20 | <10 |
| 2 | <20 | 160-320 | <10 |
| 3 | 320-1280 | 160-320 | 40-≧2560 |
| 4 | 80-640 | 160-640 | 160-1280 |
| 5 | 320-640 | 160-320 | 80-1280 |
| 6 | 160-640 | 80-320 | 80-320 |

^{a}range of titers from individual animals within each group

Protective efficacy results are summarized in Table 9 below. All animals vaccinated with a formulation containing 80E were completely protected while animals receiving NS1 alone were partially protected. Groups 3-6 had no detectable viremia, while group 2 had significantly reduced viremia compared to group 1 (control group). The control group had viremia typical of naïve hamsters challenged similarly.

TABLE 9
Protective Efficacy of ISCOMATRIX ® Adjuvanted
West Nile 80E and NS1 Formulations in Golden Hamsters

| Group | # Survivors/total | % Survival |
|---|---|---|
| 1 | 7/15 | 47 |
| 2 | 13/15^{a} | 87 |
| 3 | 15/15^{b} | 100 |
| 4 | 15/15^{b} | 100 |
| 5 | 15/15^{b} | 100 |
| 6 | 15/15^{b} | 100 |

^{a}p value = 0.022 relative to group 1 (Fisher exact probability test).
^{b}p value = 0.0011 relative to group 1 (Fisher exact probability test).

### EXAMPLE 8

### Immunogenicity and Protective Efficacy of West Nile Vaccine Candidates in Combination with Various Adjuvants in Golden Hamsters

Female golden hamsters (15 per group) were immunized, subcutaneously, with the individual vaccine formulations of 80E in combination with several adjuvants as detailed in Table 10 below. The groups of hamsters were administered adjuvant in combination with a mock antigen prepared from mock transformed Drosophila media in a manner analogous to the method used for the 80E antigen preparation. Hamsters were given a second immunization approximately 4 weeks post dose 1. Approximately 2 weeks after the second vaccination, hamsters from each group were bled and antibody titers to West Nile virus determined by hemagglutination inhibition, complement fixation, and PRNT assays as described above. Immediately after the blood samples were obtained, all hamsters were challenged by administration of 10⁴TCID₅₀ of live virus (West Nile virus strain NY 385-99). Six randomly selected hamsters from each group were bled daily for 6 days following challenge to determine the level of viremia and antibody response to viral challenge. Animals were held for 30 days following challenge for observation of morbidity and mortality. At the end of the 30 day holding period, the surviving animals were bled once more for antibody determinations, and then euthanized.

TABLE 10
Definition of Groups for Adjuvant Comparison Golden Hamster
Challenge Study

| Group | Antigen | Adjuvant |
|---|---|---|
| 1 | 1 μg 80E | ISCOMATRIX ® |
| 2 | 1 μg 80E | GPI-0100 |
| 3 | 1 μg 80E | CoVaccine HT |
| 4 | 1 μg 80E | Alum |
| 5 | 1 μg 80E | None |
| 6 | 1 μg mock | ISCOMATRIX ® |
| 7 | 1 μg mock | GPI-0100 |
| 8 | 1 μg mock | CoVaccine HT |
| 9 | 1 μg mock | Alum |
| 10 | 1 μg mock | None |

### EXAMPLE 9

### Immunogenicity and Protective Efficacy of West Nile Vaccine Candidates in Combination with Adjuvants in Non-Human Primates

Thirty six healthy, adult 3-7 kg rhesus macaques of either sex which are flavivirus antibody negative were vaccinated with the formulations described in Table 11 below by intramuscular injection of 0.5 ml into the deltoid muscle. Blood was drawn prior to administration of each dose of vaccine, and one and six months following the last dose of vaccine for analysis of humoral and cellular immunity as described in Example 5. Approximately six months after the last dose of vaccine the animals were challenged by subcutaneous administration of an appropriate dose of live virus (West Nile virus strain NY 385-99). Clinical signs were monitored for 30 days following challenge. Blood was collected from the challenged animals daily for a period of 10 days following challenge for evaluation of the level of virus in the blood (viremia). After 30 days animals were sacrificed and necropsies, including analysis for CNS pathology, were performed.

TABLE 11
Experimental Design for Rhesus Monkey Challenge Study

| | | 80E | NS1 | | | |
|---|---|---|---|---|---|---|
| | # of | Dose | Dose | | | Vaccination |
| Group | Animals | (μg) | (μg) | Adjuvant | # of Doses | Days |
| 1 | 4 | 1 | 0 | A | 4 | 0, 20, 40, 60 |
| 2 | 4 | 1 | 0 | B | 4 | 0, 20, 40, 60 |
| 3 | 4 | 1 | 1 | A | 4 | 0, 20, 40, 60 |
| 4 | 4 | 1 | 1 | B | 4 | 0, 20, 40, 60 |
| 5 | 4 | 25 | 0 | A | 4 | 0, 20, 40, 60 |
| 6 | 4 | 25 | 0 | B | 4 | 0, 20, 40, 60 |
| 7 | 4 | 25 | 10 | A | 4 | 0, 20, 40, 60 |
| 8 | 4 | 25 | 10 | B | 4 | 0, 20, 40, 60 |
| 9 | 4 | 0 | 0 | A | 4 | 0, 20, 40, 60 |
| 10 | 4 | 0 | 0 | B | 4 | 0, 20, 40, 60 |

### EXAMPLE 10

### Immunogenicity and Protective Efficacy of West Nile Vaccine Candidates in Combination with Adjuvants in Horses

Twenty healthy, adult horses (two groups of 10 horses each) of either sex which were flavivirus antibody negative were vaccinated with the test (adjuvanted 80E) or control (adjuvant plus mock antigen) formulation by intramuscular injection of 0.5 ml into the deltoid muscle. Blood was drawn prior to administration of each dose of vaccine, and one and six months following the last dose of vaccine for analysis of humoral immunity as described in Example 3. Approximately six months after the last dose of vaccine the animals were challenged by the bite of mosquitoes infected with live virus (West Nile virus strain NY 385-99). Clinical signs were monitored for 30 days following challenge. Blood was collected from the challenged animals daily for a period of 10 days following challenge for evaluation of the level of virus in the blood (viremia). After 30 days animals were sacrificed and necropsies, including analysis for CNS pathology, were performed.

### EXAMPLE 11

### Safety and Immunogenicity of West Nile Vaccine Candidate in Combination with Adjuvants in Humans

The safety and immunogenicity of the West Nile 80E vaccine candidate in combination with a relevant adjuvant was assessed in human subjects in a Phase I clinical trial. The phase 1 study design was a dose escalation study (1×, 5× and 25× dose) with a prime at time 0 followed by either 1 booster injection at day 56 or two booster injections at days 28 and 84. Safety was assessed following vaccination of each cohort and advancement to the next cohort depended on successful demonstration that the previous formulation was safe. Immunogenicity is assessed by PRNT assay before and after administration of each dose of vaccine.

### EXAMPLE 12

### Dimeric Constructions of West Nile Virus 80E to Further Increase Immunogenicity of Recombinant Subunit Protein Vaccine

Specific constructs of West Nile 80E may be prepared to facilitate formation of dimers, thereby enhancing the immunogenicity of the protein product. As disclosed in U.S. Pat. No. 6,749,857, addition of flexible linkers between tandem copies of 80E or dimerization domains appended to the carboxy terminus of the Dengue 80E molecule facilitates the formation of dimeric 80E, which has increased immunogenicity compared to monomeric 80E. Similarly, dimeric West Nile 80E molecules may be prepared. The dimeric West Nile 80E proteins may be administered to subjects in combination with several adjuvants as described in previous examples and the levels of virus neutralizing antibody induced is anticipated to be higher than in subjects administered monomeric 80E in combination with the same adjuvant.

### EXAMPLE 13

### Immunogenicity of West Nile 80E in Combination with Various Adjuvants in Aged Mice

Groups of 10 female Swiss Webster mice, 12-14 months old at initiation, were immunized twice subcutaneously at a 4 week interval with 80E or mock antigen (prepared from mock transformed Drosophila media in a manner similar to the 80E antigen) in combination with several adjuvants as detailed in Table 12 below. Serum was obtained approximately 2 weeks after the second dose and virus neutralization responses assessed as described in Example 3. Cell-mediated immune responses were also determined as described in Example 5 above.

TABLE 12
Study Groups for Adjuvant Comparison in Aged Mice

| Group | 80E Dose (μg) | Adjuvant |
|---|---|---|
| 1 | 1.0 | ISCOMATRIX ®^{a} |
| 2 | 0 | ISCOMATRIX ® |
| 3 | 1.0 | GPI-0100^{b} |
| 4 | 0 | GPI-0100 |
| 5 | 1.0 | CoVaccine HT^{c} |
| 6 | 0 | CoVaccine HT |
| 7 | 1.0 | Alum^{d} |
| 8 | 0 | Alum |
| 9 | 1.0 | None |
| 10 | 0 | None |

^{a}l2 μg per dose
^{b}250 μg per dose
^{c}50 μg per dose
^{d}3 mg per dose

### EXAMPLE 14

### Immunogenicity and Protective Efficacy of West Nile Vaccine Candidates in Combination with Various Adjuvants in Aged Golden Hamsters

Female golden hamsters (15 per group), aged 16 months, were immunized, subcutaneously, with the individual vaccine formulations of 80E in combination with several adjuvants as detailed in Example 8 above. The control groups of hamsters were administered adjuvant in combination with a mock antigen prepared from mock transformed Drosophila media in a manner analogous to the method used for the 80E antigen preparation. Hamsters were given a second immunization approximately 4 weeks post dose 1. Approximately 2 weeks after the second vaccination, hamsters from each group were bled and antibody titers to West Nile virus determined by hemagglutination inhibition, complement fixation, and PRNT assays as described above. Immediately after the blood samples were obtained, all hamsters were challenged by administration of 10⁴TCID₅₀ of live virus (West Nile virus strain NY 385-99). Six randomly selected hamsters from each group were bled daily for 6 days following challenge to determine the level of viremia and the antibody response to viral challenge. Animals were held for 30 days following challenge for observation of morbidity and mortality. At the end of the 30 day holding period, the surviving animals were bled once more for antibody determinations, and then euthanized.

### EXAMPLE 15

### Assessment of Durability of Protective Efficacy of WN 80E+/−NS1 Vaccines

Female golden hamsters (15 per group), were immunized twice subcutaneously at a 4 week interval with the individual vaccine formulations of 80E in combination with ISCOMATRIX® as detailed in the table below. The adjuvant control vaccines (groups 1, 3, and 6) were formulated to include “mock” antigen. This material was prepared by subjecting culture supernatants from induced Drosophila cells transformed with plasmids lacking the genes encoding the specific antigen to the same purification schemes used for the 80E protein. The purpose of including this material with adjuvant is to control for any possible non-specific immunostimulatory effects of potential contaminants from the cell cultures co-purified with the antigens. An amount of “mock” antigen equivalent to the amount that would be present in 1 μg of 80E+1 μg of NS1 was used.

TABLE 13
Groups for Hamster Challenge Experiment Assessing
Durability of Protection

| | # of | | | |
|---|---|---|---|---|
| group # | animals | 80E dose (μg) | NS1 dose (μg) | Adjuvant |
| 1 | 15 | 0 | 0 | ISCOMATRIX ® |
| 2 | 15 | 1 | 0 | ISCOMATRIX ® |
| 3 | 15 | 0 | 0 | ISCOMATRIX ® |
| 4 | 15 | 1 | 0 | ISCOMATRIX ® |
| 5 | 15 | 1 | 1 | ISCOMATRIX ® |
| 6 | 15 | 0 | 0 | ISCOMATRIX ® |
| 7 | 15 | 1 | 0 | ISCOMATRIX ® |
| 8 | 15 | 1 | 1 | ISCOMATRIX ® |

Immune response analysis included assessment of PRNT, CF, and HI titers two to three weeks after the second dose of vaccine in all animals. Animals from groups 3-8 were further tested for humoral immune responses using the PRNT, CF, and HI test 6 months following the final dose and groups 6-8 were similarly tested 12 months following the final dose of vaccine.

Protective efficacy was assessed by challenge using a standard dose of WN virus as described in Example 14. Groups 1-2 were challenged 2-3 weeks following the final dose of vaccine, Groups 3-5 were challenged 6 months following the final dose of vaccine, and groups 6-8 were challenged 12 months following the final dose of vaccine. In each case 6 randomly selected hamsters from each challenged group of 15 animals were bled daily for 6 consecutive days to determine the level of viremia and antibody response (by HI titer). All challenged animals were held for 30 days after viral challenge and observed for morbidity and mortality. After the 30 day holding period, all challenged surviving animals were bled for antibody titers and then euthanized.

### EXAMPLE 16

### Assessment of Durability of Protective Efficacy of WN 80E+/−NS1 Vaccines With Different Adjuvants

Female golden hamsters (15 per group), were immunized twice, subcutaneously, at a 4 week interval with the individual vaccine formulations of 80E in combination with various adjuvants as detailed in the table below. The adjuvant control vaccines (groups 3, 6, and 9) were formulated to include “mock” antigen as described in Example 15 above.

TABLE 14
Groups for Hamster Challenge Study Assessing Durability of Protective
Response in Animals Vaccinated with Formulation Based on Different
Adjuvants

| Group | 80E Dose (μg) | NS Dose (μg) | Adjuvant |
|---|---|---|---|
| 1 | 1.0 | 0 | Alum^{a} |
| 2 | 1.0 | 1.0 | Alum |
| 3 | 0 | 0 | Alum |
| 4 | 1.0 | 1.0 | GPI-0100^{b} |
| 5 | 1.0 | 0 | GPI-0100 |
| 6 | 0 | 0 | GPI-0100 |
| 7 | 1.0 | 0 | CoVaccine HT^{c} |
| 8 | 1.0 | 1.0 | CoVaccine HT |
| 9 | 0 | 0 | CoVaccine HT |
| 10 | 1.0 | 1.0 | None |

1) ^{a}3 mg Al(OH)₃per dose
2) ^{b}250 μg per dose
3) ^{c}50 μl per dose

Twelve months after the booster vaccinations, all animals were bled. Serum samples were assayed for HI, CF, and PRNT titers to West Nile virus. After the blood samples were obtained each hamster was challenged with the standard dose of WN virus. Six randomly selected hamsters from each challenged group of 15 animals were bled daily for 6 consecutive days to determine the level of viremia and antibody response (by HI titer). All challenged animals were held for 30 days after viral challenge and observed from morbidity and mortality. After the 30 day holding period, all challenged surviving animals were bled for antibody titers and then euthanized.

### EXAMPLE 17

### Immunogenicity and Protective Efficacy of West Nile Vaccine Candidates in Combination with Various Adjuvants in Young (Weanling Golden Hamsters

Female golden hamsters (15 per group), aged 3 weeks (after weaning), were immunized twice at a 4 week interval subcutaneously with the individual vaccine formulations of 80E in combination with several adjuvants as detailed in Example 8 above. The control groups of hamsters were administered adjuvant in combination with a mock antigen prepared from mock transformed Drosophila media in a manner analogous to the method used for the 80E antigen preparation. Hamsters were given a second immunization approximately 4 weeks post dose 1. Approximately 2 weeks after the second vaccination, hamsters from each group were bled and antibody titers to West Nile virus determined by hemagglutination inhibition, complement fixation, and PRNT assays as described above. Immediately after the blood samples were obtained, all hamsters were challenged by administration of 10⁴TCID₅₀ of live virus (West Nile virus strain NY 385-99). Six randomly selected hamsters from each group were bled daily for 6 days following challenge to determine the level of viremia and the antibody response to viral challenge. Animals were held for 30 days following challenge for observation of morbidity and mortality. At the end of the 30 day holding period, the surviving animals were bled once more for antibody determinations, and then euthanized.

### EXAMPLES 18-20

### Protective Efficacy of Adjuvanted West Nile 80E+/−NS1 Vaccines in Immunocompromised Animals

### EXAMPLE 18 Protection of Leukopenic Mice and Hamsters

As discussed above, iatrogenic immunodeficiency is necessarily induced in particular groups of patients such as cancer patients undergoing cytotoxic chemotherapy. Such patients are at significantly increased risk of severe disease if exposed to West Nile virus. If such patients could be successfully immunized prior to undergoing such therapy, then this risk may be limited or managed. To simulate such conditions in an animal model of disease, mice or hamsters are vaccinated and then rendered leukopenic by cyclophosphamide administration (Lieberman, M M and Frank, W J, J. Surg. Res. (1988) 44: 242) prior to challenge with live virus. Control animals are not vaccinated (administered only “mock” antigen and adjuvant), then rendered leukopenic, and challenged with virus. The adjuvants used were those listed under Example 13 above. Vaccines formulated with West Nile 80E+/−NS1 were tested. Survival of vaccinated and control groups after challenge were compared to determine the protective efficacy of vaccination in this animal model.

### EXAMPLE 19

### Protection of Complement Deficient Mice

DBA/2J mice have a primary deficiency in the fifth component (C5) of the classical complement system (Cerquetti, M C, et al., Infect. Immun. (1983) 41: 1017). C5 is essential for the formation of the membrane attack complex (C5b-C9) by either the classical or alternative pathways of complement activation. This animal model thus simulates a primary immunodeficiency which may be important in humoral immunity to flaviviral infection. DBA/2J mice were vaccinated with West Nile 80E+/−NS1 vaccines formulated with the adjuvants listed in Example 13 above. Adjuvant control groups of mice were included as above. Mice were then challenged with live West Nile virus and survival of vaccinated and adjuvant control animals compared to determine protective efficacy.

### EXAMPLE 20

### Protection of T Cell Deficient Mice

Swiss Nude mice are deficient in T cells and thus serve as a model for either a primary T cell deficiency (e.g., DiGeorge Syndrome), or a secondary (acquired) T cell deficiency, such as Acquired Immune Deficiency Syndrome (AIDS) secondary to HIV infection. Swiss Nude mice were vaccinated with West Nile 80E+/−NS1 vaccines formulated with the adjuvants listed in Example 13 above. Adjuvant control groups of mice were included as above. Mice were challenged with live West Nile virus and survival of vaccinated and adjuvant control animals compared to determine protective efficacy.

### EXAMPLE 21

### Use of Recombinant WN 80E for Design and Screening of Antiviral Candidate Small Molecules

Purified WN 80E was used for crystallization of the dimeric and trimeric forms of the envelope, assay development, and preliminary binding studies (co-crystallization, virtual screening, and combinatorial studies) for design and screening of candidate small molecule anti-virals. Crystallization trials were conducted using numerous conditions (e.g. combinations of salt, organic polymers, alcohols, detergents, buffers in a wide range of pH, temperature, etc.). Conditions similar to those used for crystallization of DEN 80% E and TBE 80E were evaluated as these two proteins have a high degree of identity with the West Nile protein. Additional conditions were determined through sparse matrix screens. These initial crystallization screens were done in sitting drop, hanging drop, sandwich drop, batch, under oil, or in gel format using a combination of protein and crystallant solution. Conditions that yielded crystals were optimized to yield diffraction quality crystals using grid screens, where the various components of the crystallant solution were systematically optimized. High resolution data were collected at a synchrotron radiation source. The data were scaled and integrated using software such as HKL2000 (Otwinowski Z. Processing of X-ray Diffraction Data Collected in Oscillation Mode. Volume 276. New York: Academic Press; (1997) pp 307-326). The previously solved structures of the dengue and TBE envelope proteins and the NMR model of West Nile envelope domain III were used as molecular replacement models. Additional phasing was done, as necessary, using multiple isomorphous replacement (MIR), multiple anomalous dispersion (MAD), or single anomalous dispersion (SAD) methods. A protein model was built into electron density using a program such as O (T. A. Jones, M. Kjeldgaard, “Essential O', software manual, Uppsala 1998) and refined using a program such as CNS (Brunger A. et al., Acta Crystallogr D Biol Crystallogr (1998) 54:905-921). Protein physical chemistry, including bond length and angle was monitored through software such as “Procheck” (Laskowski R. et al., J. Appl. Cryst. 26:283-291.).

The crystal structures derived from the WN 80E were used for screening of potential small molecule antiviral compounds through combinatorial, virtual, and co-crystal libraries. Compounds were derived from available commercial libraries and the structures of these compounds were visually inspected for verification of lead-like properties (lack of bio-reactive groups, accessibility to further chemistry at multiple points, etc.). These compounds were screened in silico, using software such as FlexX from Tripos, with the highest scoring compounds being purchased for assay. For co-crystal screening, the compound library was sub-grouped into groups of 5 to 15 compounds based on diversity that allowed individual compounds to be identified in a crystallographic electron density map. The screening of these chemical cocktails with diverse scaffolds and unique atoms or reactive groups facilitated identification of a specific molecule in the crystal structure. The co-crystallization library was used to screen West Nile envelope crystals and confirm presence of compounds binding at a site of interest or a potentially novel site of cell entry inhibition. Controls were included in the screens, including non-drug like controls such as β-Octyl Glucoside, which has been shown to bind the hinge region of the dengue envelope.

Purified WN 80E protein was also used to develop an assay that identifies molecules which inhibit either the early or late stages of envelope mediated fusion (following the formation of the trimeric fusion intermediate form of the E protein, thereby blocking viral insertion). In early stage fusion, two methods were used to measure the conformational changes which occur. The first method is to utilize a fluorescent tag on the protein itself. As the proteins initiate fusion, a percentage of the fluorescent tags become buried internally and the total fluorescence decreases. The second method utilized the addition of a fluorescent molecule, bis-ANS, to the solution. Bis-ANS binds to hydrophobic regions of the molecule. Again, as the molecules begin to trimerize, the percent of hydrophobic regions available is reduced and the total fluorescence in the solution was modulated. Detection of trimerization for both these methods is measured by prompt fluorescence. For assessment of impact on the late stages of envelope mediated fusion, a different assay was used. This assay allowed the measurement of (i) binding of the stem portion of the protein compared to (ii) inhibition of binding of the stem Protein through the use of a fluorescently labeled synthetic peptide derived from the stem region of the envelope protein. Detection of binding of this fluorescent peptide to trimer protein was measured by fluorescence polarization.

1. An immunogenic composition comprising:
an effective amount of at least one purified West Nile virus envelope (“E”) polypeptide, lacking all or a portion of its membrane spanning domain such that said E polypeptide is secretable into growth medium when expressed recombinantly in a host cell; and
an effective amount of one or more immunomodulating agents selected from the group comprising saponin, saponin-based adjuvant, aluminum-based adjuvant, and emulsion-based adjuvant, wherein the immunogenic composition induces the production of neutralizing antibodies and a cell-mediated immune response from a host provided with the immunogenic composition.

2. An immunogenic composition comprising:
an effective amount of at least one purified West Nile virus envelope (“E”) polypeptide, wherein the E polypeptide constitutes approximately 80% of the length of wild type E starting from amino acid residue 1 at its N-terminus, such that said E polypeptide is secretable into growth medium when expressed recombinantly in a host cell; and
an effective amount of one or more immunomodulating agents selected from the group comprising saponin, saponin-based adjuvant, aluminum-based adjuvant, and emulsion-based adjuvant, wherein the immunogenic composition induces the production of neutralizing antibodies and a cell-mediated immune response from a host provided with the immunogenic composition.

3. An immunogenic composition comprising:
an effective amount of at least one purified West Nile virus envelope (“E”) polypeptide, wherein the polypeptide has the amino acid sequence set forth in SEQ ID:1, which E polypeptide is secretable into growth medium when expressed recombinantly in a host cell; and
an effective amount of one or more immunomodulating agents selected from the group comprising saponin, saponin-based adjuvant, aluminum-based adjuvant, and emulsion-based adjuvant, wherein the immunogenic composition induces the production of neutralizing antibodies and a cell-mediated immune response from a host provided with the immunogenic composition.

4. The immunogenic composition of claim 1, 2, or 3, wherein the E polypeptide is recombinantly produced and expressed in insect host cells.

5. The immunogenic composition of claim 1, 2, or 3, wherein the E polypeptide is recombinantly produced and expressed in Drosophila melanogaster Schneider 2 (“S2”) host cells.

6. The immunogenic composition of claim 1, 2, or 3, further comprising at least one recombinant West Nile virus non-structural protein.

7. The immunogenic composition of claim 1, 2, or 3, further comprising recombinant West Nile virus non-structural protein 1 (“NS1”) produced and expressed in Drosophila melanogaster Schneider 2 (“S2”) cell lines.

8. The immunogenic composition of claim 1, 2, or 3, wherein the saponin-based adjuvant is ISCOMATRIX® or GPI-0100.

9. The immunogenic composition of claim 1, 2, or 3, wherein the emulsion-based adjuvant is Co-Vaccine HT.

10. The immunogenic composition of claim 1, 2, or 3, further comprising a pharmaceutically acceptable excipient.

11. An immunoprotective composition comprising the immunogenic composition of claim 1, 2, or 3.

12. The immunogenic composition of claim 1, 2, or 3 for use in immunodeficient populations.

13. A composition for use in the development of antiviral compositions comprising crystallized E polypeptide of claim 1, 2, or 3, wherein crystal structures of the E polypeptide are used to screen potential small molecule anti-West Nile viral compounds through libraries selected from the group comprising combinatorial, virtual, and co-crystal.

14. A composition for use in the development of antiviral compositions comprising E polypeptide of claim 1, 2, or 3, wherein purified E polypeptide is used in an assay that identifies molecules that block envelope mediated fusion required viral insertion.

15. A kit useful for providing immune protection for West Nile virus comprising a container containing an immunogenic composition of claim 1, 2, or 3 and a pharmaceutically acceptable carrier.

16. A composition of antibodies consisting essentially of antibodies generated in a mammalian subject administered an immunogenic amount of the vaccine of claim 1, 2, or 3.

17. A method for raising an immunogenic response from a host, comprising administering in a therapeutically acceptable manner a therapeutically effective amount of the immunogenic compostion of claim 1, 2, or 3.

18. A method of treating West Nile infection, comprising administering to a subject having, or at risk of having, West Nile infection an effective amount of the immunogenic composition of claim 1, 2, or 3, thereby treating the infection.

19. A method of providing immune protection against West Nile comprising administering to a subject in need of protection an effective amount of the immunogenic composition of claim 1, 2, or 3, thereby providing protection from West Nile infection.

20. A method for preparing an immunogenic composition for treatment of West Nile comprising:
(a) forming an immunogenic composition of claim 1, 2, or 3;
(b) providing a suitable excipient; and
(c) mixing the immunogenic composition of (a) with the excipient of (b).

21. A method of producing anti-West Nile antibodies comprising administering to a mammal an effective amount of an E polypeptide of claim 1, 2, or 3.

22. A method of detecting the presence of West Nile virus in a sample comprising contacting the sample with an antibody of claim 21 and detecting binding of the antibody to the E polypeptide, wherein formation of a complex between the antibody and the E polypeptide is indicative of the presence of West Nile in the sample.

23. The method of claim 22, wherein the antibody is detectably labeled.

24. A method of detecting West Nile infection comprising contacting a biological sample with the E polypeptide of claim 1, 2, or 3 under conditions which allow formation of an antibody-antigen complex and detecting said complex.

25. The method of claim 24, wherein the E polypeptide antigen is detectably labeled.

26. A polyclonal antibody composition made by producing anti-West Nile antibodies by administering to a mammal an effective amount of an immunogenic composition of claim 1, 2, or 3.

27. A purified antibody that specifically binds to a polypeptide of claim 1, 2, or 3.

28. An isolated polynucleotide encoding the West Nile virus E polypeptide of claim 1, 2, or 3.

29. An isolated polynucleotide having the nucleotide sequence set forth in SEQ ID:3.

30. An isolated polynucleotide, wherein the nucleotide sequence encodes the polypeptide with amino acid sequence as set forth in SEQ ID:1.

31. An immunodiagnostic for the detection of West Nile virus, wherein said immunodiagnostic comprises the E polypeptide of claim 1, 2, or 3.

32. The immunodiagnostic of claim 31, further comprising West Nile nonstructural protein 1 (“NS1 ”).

33. The immunodiagnostic of claim 32, wherein the NS1 has been produced and expressed in Drosophila melanogaster Schneider 2 (“S2”) host cells.

34. An immunodiagnostic kit for the detection of Flavivirus in a test subject, comprising:
a) the E polypeptide defined in claim 1, 2, or 3;
b) a suitable support phase coated with the E polypeptide; and
c) labeled antibodies immunoreactive to anti-West Nile antibodies from said test subject.

35. An immortalized B cell line, wherein B cells have been generated in response to the administration to a mammalian subject of an immunogenic amount of the immunogenic composition of claim 1, 2, or 3 and secrete antibodies in response to said immunogenic composition.

36. A hybridoma, wherein the B cells of claim 35 have been fused with nonsecretory myeloma cells.


SEQUENCE LISTING (16 sequences)

SEQ ID NO: 1 - DNA, 20 bp, Artificial Sequence
  oligodeoxyribonucleotide
tccatgacgttcctgacgtt

SEQ ID NO: 2 - DNA, 20 bp, Artificial Sequence
  oligodeoxyribonucleotide
ataatcgacgttcaagcaag

SEQ ID NO: 3 - DNA, 20 bp, Artificial Sequence
  oligodeoxyribonucleotide
ataatagagcttcaagcaag

SEQ ID NO: 4 - DNA, 21 bp, Artificial Sequence
  oligodeoxyribonucleotide
tccaatgagcttcctgagtct

SEQ ID NO: 5 - DNA, 6 bp, Artificial Sequence
  oligodeoxyribonucleotide
gacgtt

SEQ ID NO: 6 - DNA, 20 bp, Artificial Sequence
  oligodeoxyribonucleotide
ggtgcatcgatgcagggggg

SEQ ID NO: 7 - DNA, 20 bp, Artificial Sequence
  oligodeoxyribonucleotide
ggtgcatcgatgcagggggg

SEQ ID NO: 8 - DNA, 27 bp, Artificial Sequence
  oligodeoxyribonucleotide
tcgtcgctgtctccgcttcttcttgcc

SEQ ID NO: 9 - DNA, 24 bp, Artificial Sequence
  oligodeoxyribonucleotide
tcgtcgttttgtcgttttgtcgtt

SEQ ID NO: 10 - DNA, 20 bp, Artificial Sequence
  oligodeoxyribonucleotide
atcgactctcgagcgttctc

SEQ ID NO: 11 - DNA, 12 bp, Artificial Sequence
  oligodeoxyribonucleotide
tcgagcgttctc

SEQ ID NO: 12 - DNA, 22 bp, Artificial Sequence
  oligodeoxyribonucleotide
tgactgtgaacgttcgagatga

SEQ ID NO: 13 - protein, 25 aa, Flavivirus
GGGSGGGGSGGGTGGGSGGGSGGGG

SEQ ID NO: 14 - protein, 55 aa, Flavivirus
GGGSGGGGSGGGTGGGSGGGSPRMKQLEDKVEELLSKNYHLENEVARLKKLVGER

SEQ ID NO: 15 - protein, 60 aa, Flavivirus
GGGSGGGGSGGGTGGGSGGGSPRMKQLEDKVEELLSKNYHLENEVARLKKLVGERGGCGG

SEQ ID NO: 16 - protein, 59 aa, Flavivirus
GGGSGGGGSGGGTGGGSGGGSPGELEELLKHLKELLKGPRKGELEELLKHLKELLKGEF